(19) United States
(12) Patent Application Publication
(10) Pub. No.: US 2024/0252719 A1
(43) Pub. Date: Aug. 1, 2024

(54) SYSTEMS AND METHODS FOR IN VIVO CELL REPLACEMENT

(71) Applicant: PhosPrint P.C., Athens (GR)

(72) Inventors: Ioanna Zergioti, Athens (GR); Apostolos Klinakis, Attica (GR); Panagiotis Karakaidos, Attika (GR)

(21) Appl. No.: 18/425,982

(22) Filed: Jan. 29, 2024

Related U.S. Application Data
(60) Provisional application No. 63/442,043, filed on Jan. 30, 2023.

(51) Int. Cl. A61L 27/36; A61F 2/04; A61L 27/38; B33Y 10/00; B33Y 70/00; B33Y 80/00

(52) U.S. Cl. CPC A61L 27/3679; A61F 2/042; A61L 27/3616; A61L 27/3629; A61L 27/3687; A61L 27/3691; A61L 27/3834; A61L 27/3882; B33Y 10/00; B33Y 70/00; B33Y 80/00; A61F 2240/002; A61F 2250/0081

(57) ABSTRACT

A method for in-vivo cell replacement is disclosed. The method includes steps of extracting donor cells from a donor tissue of a patient; generating donor material including the donor cells; removing one or more cell layers from a receiver tissue of the patient to expose a target layer of the receiver tissue while maintaining operation of a vasculature of the receiver tissue; and printing the donor material onto the target layer of the receiver tissue. A tissue position assembly, a composition for use in a treatment of bladder damage or bladder dysmorphism, and a system for supplying donor material to a bioprinter are also disclosed.

### CROSS-REFERENCE TO RELATED APPLICATION

The present application claims the benefit under 35 U.S.C. § 119(e) of U.S. Provisional App. No. 63/442,043 filed by Zergioti on Jan. 30, 2023, entitled “SYSTEMS AND METHODS FOR IN-VIVO CELL REPLACEMENT”, which is incorporated herein by reference in its entirety.

### TECHNICAL FIELD

The present invention generally relates to the fields of cell biology and tissue engineering, and, more particularly, to a system and method of modifying and generating biological tissues for transplantation. Tissue reconstruction is the science of using cells and other supporting material to generate and grow tissues and organs, often for use in transplantation. Cells from donor tissues may be isolated and repositioned on an artificial or biological substrate. The newly formed tissue may then be transplanted into the body. Tissue engineering requires considerable skill to ensure that the building of cells and supporting material upon the substrate is performed competently. Biological substrates, such as intestinal tissue derived from a transplant recipient, are often utilized in tissue engineering procedures as they are autologous and thus tolerated by the immune system. However, the use of intestinal tissue as a substrate for a non-intestinal tissue-engineered transplant, such as urinary bladder can be problematic due to the absorptive and secretive nature of intestinal tissue. Thus, it is desirable to provide a method and product that avoids the shortcomings of conventional approaches.

### SUMMARY

In some aspects, the techniques described herein relate to a method for in-vivo cell replacement including: extracting donor cells from a donor tissue of a patient; generating donor material including the donor cells; removing one or more cell layers from a receiver tissue of the patient to expose a target layer of the receiver tissue while maintaining operation of a vasculature of the receiver tissue; and printing the donor material onto the target layer of the receiver tissue.

In some aspects, the techniques described herein relate to an augmented bladder including: a first portion of a bladder of a patient; and a graft formed from intestinal tissue of the patient with undisturbed vasculature, wherein the intestinal tissue includes a layer of donor material including urothelial cells from the patient printed by steps of: extracting donor cells from a donor tissue of a patient; generating the donor material including the donor cells; removing one or more cell layers from the intestinal tissue of the patient to expose a target layer of the intestinal tissue while maintaining operation of a vasculature of the intestinal tissue; and printing the donor material onto the target layer of the intestinal tissue.

In some aspects, the techniques described herein relate to a tissue position assembly including: a rail formed from a sterilizable material, wherein the rail is configured to connect to a replaceable receiver assembly, the replaceable receiver assembly further configured to secure and position a tissue of a patient while maintaining operation of vasculature of the tissue, wherein the rail is further configured to connect to a replaceable donor assembly, the replaceable donor assembly configured to secure and position a donor plate with donor material on a surface facing the replaceable receiver assembly; and a housing connected to the rail, wherein the housing is configured to secure one or more lenses configured to focus laser light from a laser source onto the donor plate to transfer a portion of the donor material to receiver material.

In some aspects, the techniques described herein relate to a composition for use in a treatment of bladder damage or bladder dysmorphism including: donor material containing cells suitable for transfer from a donor substrate to receiver material via a bioprinter; media suitable for transfer of the donor material from the donor substrate to the receiver material; and the donor substrate loaded with the donor material and the media.

In some aspects, the techniques described herein relate to a system for supplying donor material to a bioprinter including: a donor frame; an automated stage configured to move the donor frame along an X-axis and a Y-axis; a dispenser subsystem including a pump and configured to deliver transport donor material from a reservoir to a material feeding head; one or more controllers communicatively coupled to the automated stage and the dispenser subsystem, the one or more controllers including one or more processors configured to execute a set of program instructions stored in a memory, the set of program instructions configured to cause the one or more processors to: operate the pump, wherein operating the pump transports donor material from the reservoir through the material feeding head; and operate the automated stage to move the donor frame in coordination with an operation of the pump, wherein the donor material is transported to a designated area via a movement of the donor frame and the operation of the pump.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims. In the drawings:

FIG. 1A illustrates a flowchart for a method of bioprinting, in accordance with one or more embodiments of the disclosure.

FIG. 1B illustrates a flowchart for a method of bioprinting, in accordance with one or more embodiments of the disclosure.

FIG. 2 illustrates a block diagram of a bioprinting system, in accordance with one or more embodiments of the disclosure.

FIG. 3 illustrates the composition of cells in a bladder biopsy, a microsurgical isolation of bladder mucosa, and an expansion of basal cells, in accordance with one or more embodiments of the disclosure.

FIG. 4 illustrates a system for laser ablation of cells, in accordance with one or more embodiments of the disclosure.

FIG. 5 illustrates a system for laser-induced forward transfer of cells, in accordance with one or more embodiments of the disclosure.

FIG. 6 illustrates a perspective view of a donor supply sub-system for a tissue reconstruction system, in accordance with one or more embodiments of the disclosure.

FIG. 7A illustrates an exploded view of a tissue position assembly, in accordance with one or more embodiments of the disclosure.

FIG. 7B illustrates a perspective view of a tissue position assembly integrated into a bioprinting laser, in accordance with one or more embodiments of the disclosure.

FIG. 8 illustrates schematic representations of bladder histology, isolated urothelium, and expanded basal cells, in accordance with one or more embodiments of the disclosure. The urothelium is comprised of three distinct cell types in layers of different thicknesses among species. However, umbrella cells are always on the top facing the lumen, intermediate cells in the middle layer, and basal cells) comprise the bottom layer of the urothelium. Beneath the urothelium lies lamina propria i.e. a stromal tissue, which harbors several cell types (including fibroblasts and immune cells), and intense extracellular mesh (fibrous connective tissue including several types of collagen). The outermost bladder tissue consists of smooth muscle cells (detrusor muscle), which is also called the bladder wall.

FIG. 9 illustrates a detubularized and denuded porcine intestinal segment, after suturing in “S” shape, for complete enterocystoplasty, in accordance with one or more embodiments of the disclosure. A 40 cm porcine intestinal segment (30 cm away from the ileocecal valve) was isolated with its mesentery intact. Following detubularization, the intestinal epithelium is peeled off. The remaining tissue was positioned flat in an “S” shape and the opposing ends were sutured. The image depicts the resultant neobladder graft.

FIG. 10 illustrates a schematic representation of laser-induced forward transfer (LIFT) printing of porcine primary urothelial cells (pUCs) on glucosaminoglycan (GAG) scaffold, in accordance with one or more embodiments of the disclosure. GAG is placed on a mold (not depicted) and irradiated to become photopolymerized. The scaffold is then cellularized through LIFT printing of pUCs. Then the pUC-GAG composite may be cultured ex vivo until the pUCs proliferate and differentiate towards stratified urothelium or used immediately for enterocystoplasty.

FIG. 11 illustrates a schematic representation of the proposed neobladder formation methods through tissue transdifferentiation applications, in accordance with one or more embodiments of the disclosure.

FIG. 12 illustrates a tissue section of in vivo printed denuded intestinal segment with porcine primary urothelial cells (pUCs) two weeks after augmentation cystoplasty, in accordance with one or more embodiments of the disclosure. The pUCs utilized were previously labeled through adenoviral infection of a green fluorescent protein (GFP)-expressing vector. The image depicts an immunofluorescence analysis of two distinct areas of the graft where urothelial and stromal cells were still expressing GFP and keratins (KRT5 and KRT14). Nuclei were counterstained with DAPI.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to tissue reconstruction during a surgical procedure using in-vivo and/or ex-vivo techniques. For example, some embodiments of the present disclosure are directed to the preparation of both a receiver material (e.g., a graft) formed from patient tissue and/or an artificial support as well as donor material (e.g., cells of a desired type), followed by bioprinting of the donor material onto the receiver material during a surgical procedure. As a non-limiting illustration, urothelial cells may be printed on a graft suitable for augmenting a bladder or urethra during a surgical process. Thus, tissue reconstruction may be used in the treatment of a broad range of congenital-, disease- and trauma-related organ damage, organ dysmorphism, tissue damage, and tissue dysmorphism.

Some embodiments of the present disclosure are directed to systems and methods for replacement of cells of a biological tissue with additional cells of a different type. Such tissues may be suitable for transplantation (e.g., as grafts) in a wide variety of processes. It is contemplated herein that it may be desirable to utilize biological tissues of one type from a patient to prepare a graft for an additional type of tissue in the patient, but that the desired tissues may include various cells or layers of cells that are not desirable when transplanted. In some embodiments, undesired cells from a biological tissue are removed to expose a target layer of the receiver material (e.g., without disrupting the vasculature of the receiver material/tissue) followed by printing cells of a different type onto the tissue. In this way, the target layer of the tissue may provide desirable mechanical properties and may support the growth of the new cells.

As one illustrative embodiment, systems and methods disclosed herein may be suitable for enterocystoplasty (e.g., bladder augmentation), urethroplasty or orthotopic neobladder procedures. For example, enterocystoplasty and orthotopic neobladder procedures use an autologous intestinal segment to increase bladder capacity and substitute damaged bladder tissue, respectively. The muscular part of intestine is sufficient to provide the mechanical competence required for urine storage. However, intestinal epithelium is completely incompatible with urine-blood barrier function of the urinary bladder, one of the key functions of the urinary bladder. Enterocystoplasty and orthotopic neobladder is often accompanied by severe complications in the vast majority (e.g., up to 94%) of the cases. In some embodiments, epithelial cells are removed from a portion of an intestine to expose a target layer (e.g., a muscular layer) of the intestine followed by printing urothelial cells onto the target layer. In this way, the undesired epithelial cells naturally present on the intestine may be replaced by desirable urothelial cells suitable for the bladder. It is further contemplated herein that a similar technique may be applied for urethroplasty procedures.

It is contemplated herein that undesired cells on a receiver material formed from a biological tissue (e.g., intestinal tissue, or the like) may be removed using a variety of techniques within the spirit and scope of the present disclosure. In some embodiments, undesired cells are removed from a biological receiver material using laser ablation. For example, removal of undesired cells from biological receiver materials is described generally in U.S. patent application Ser. No. 17/387,801 filed Jul. 28, 2021, to which the present disclosure claims priority and which is incorporated herein by reference in its entirety. In this configuration, one or more laser pulses may selectively ablate undesired cells from the receiver material to expose a target layer without damaging the target layer. Continuing the bladder augmentation and orthotopic neobladder examples, one or more laser pulses may selectively ablate epithelial cells or the like from intestinal tissue to expose a muscular layer prior to bioprinting of urothelial cells. In some embodiments, the undesired cells are removed using mechanical techniques (e.g., scraping, peeling, rubbing, or the like). In some embodiments, the undesired cells are removed using chemical techniques (e.g., solvents, or the like).

It is further contemplated herein that desired cells (e.g., cells of a desired type such as, but not limited to, urothelial cells) may be printed on a receiver material using a variety of techniques within the spirit and scope of the present disclosure. In some embodiments, desired cells are printed using a laser-induced forward transfer (LIFT) technique. For example, the use of LIFT to print donor material is also described generally in U.S. patent application Ser. No. 17/387,801 filed Jul. 28, 2021, to which the present disclosure claims priority and which is incorporated herein by reference in its entirety. In some embodiments, desired cells are printed using inkjet techniques. In some embodiments, desired cells are printed using extrusion techniques.

Additionally, desired cells may be printed onto a receiver material using in-vivo or ex-vivo techniques. For example, an in-vivo technique may include in-situ printing the desired cells onto the receiver material formed from a biological tissue derived from the patient when the receiver material is at least partially attached to the body either before or after transplantation. As another example, an ex-vivo technique may include printing the desired cells onto the receiver material when the receiver material is outside the body and subsequently attaching the receiver material to the desired tissue (e.g., the bladder, urethra, or the like). In this case, the receiver material may include a biological tissue derived from the patient or another source or may include an artificially engineered substrate such as, but not limited to, a glucosaminoglycan (GAG) scaffold.

As used herein, an in-vivo technique is a technique that is performed within the body of a living subject (e.g., an animal (non-human animal), mammal, or human). As used herein, an ex-vivo technique is a technique that is performed outside of the body of the living subject.

Some embodiments of the present disclosure are directed to methods for tissue reconstruction using in-vivo and ex-vivo techniques. Some embodiments of the present disclosure are directed to grafts suitable for tissue reconstruction using in-vivo and ex-vivo techniques. Some embodiments of the present disclosure are directed to systems suitable for performing or facilitating tissue reconstruction using in-vivo and ex-vivo techniques. Some embodiments of the present disclosure are directed to tissue reconstruction using techniques that involve removing a target layer of receiver tissue while maintaining operation of the vasculature of the receiver tissue. Some embodiments of the present disclosure are directed to an altered or augmented organ, such as an augmented bladder that includes intestinal tissue from the patient with undisturbed vasculature along with urothelial cells that have been printed onto the augmented bladder.

Some embodiments of the present disclosure are directed to a tissue position assembly for use in a bioprinting protocol. The tissue position assembly holds both a plate donor reservoir for holding cells and a plate receiver for holding the material being printed upon. The tissue position assembly simplifies the process of securing the donor and receiver material during the bioprinting process.

Some embodiments of the present disclosure are directed to a composition for use in the treatment of organ damage or organ dysmorphism (e.g., a condition in which part of the body is a different shape from normal). The composition may include cells, media, and/or a donor substrate loaded with the cells and media.

Some embodiments of the present disclosure are directed to a system for supplying donor material to a bioprinter. The system includes a donor frame, and an automated stage for moving the donor frame, and a dispenser subsystem for transporting donor material.

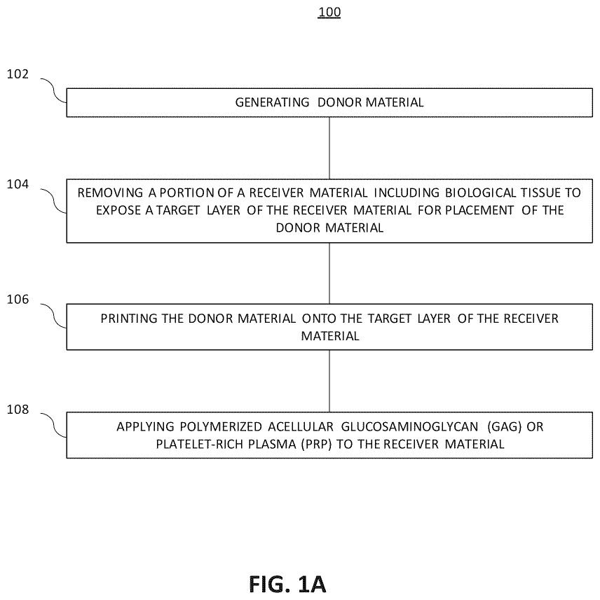

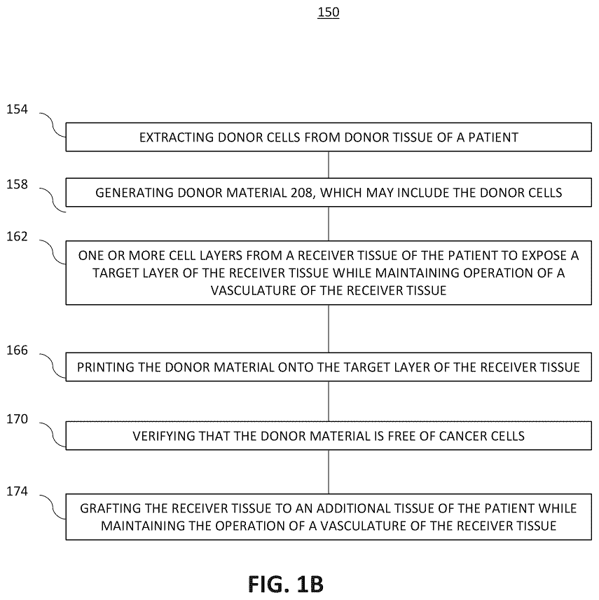

Referring now to FIGS. 1-12, systems and methods for tissue reconstruction using in-vivo and ex-vivo techniques are described in greater detail, in accordance with one or more embodiments of the present disclosure.

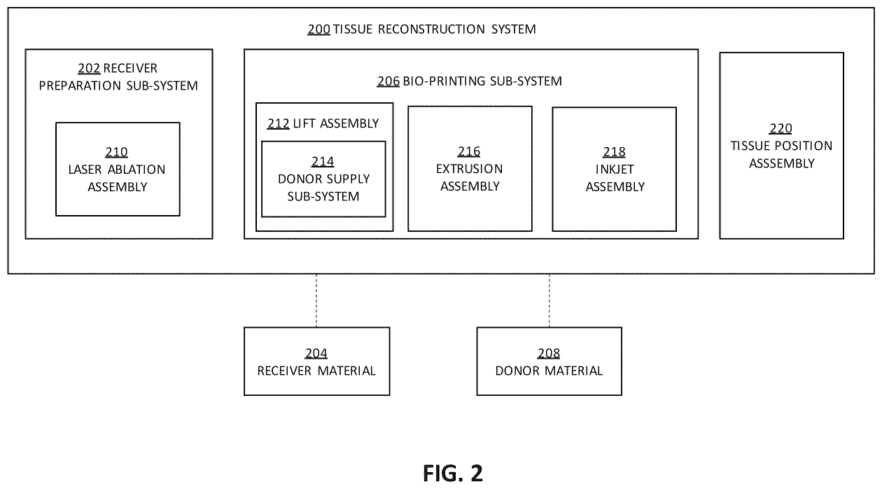

FIG. 1A is a block diagram of a method 100 for tissue reconstruction, in accordance with one or more embodiments of the present disclosure. FIG. 2 is a block diagram view of a tissue reconstruction system 200 for tissue reconstruction, in accordance with one or more embodiments of the present disclosure. It is contemplated herein that that the system 200 may be suitable for implementing one or more steps of the method 100, but is not limited to performing steps of the method 100. Further, the method 100, or any steps thereof, are not limited by the description of the system 200 and may be performed using any suitable components or combinations of components.

Referring to FIG. 2, a brief description of the system 200 is provided, in accordance with one or more embodiments of the present disclosure. In some embodiments, the system 200 includes a receiver preparation sub-system 202 for preparing receiver material 204 to receive donor material 208 via a bio-printing process. The receiver preparation sub-system 202 may include one or more components suitable for mechanically securing and/or positioning a receiver material 204 such as, but not limited to, a platform, clamps, forceps, clips, or sutures. For example, the receiver preparation sub-system 202 may include components suitable for securing the receiver material 204 in a substantially flat position required for one or more processing steps (e.g., steps of the method 100). Further, in the case that the receiver material 204 includes biological tissue from a patient undergoing surgery, the receiver preparation sub-system 202 may mechanically secure and/or position the receiver material 204 while the receiver material 204 is connected to the patient such that one or more processing steps (e.g., steps of the method 100) may be performed during surgery.

In some embodiments, the receiver preparation sub-system 202 includes one or more components suitable for removing one or more cells (or layers of cells) from the receiver material 204 to expose a target layer of the receiver material 204. For example, a receiver material 204 may include both desired layers (e.g., muscular layers, or the like) providing desired properties for transplantation and undesirable cells or layers that are not suitable for transplantation. In the example of bladder augmentation, a receiver material 204 including intestinal tissue may include muscular layers and the like that provide mechanical stability and biocompatibility, and also include epithelial cells that are incompatible with the urine-blood barrier function of the bladder. Accordingly, the receiver preparation sub-system 202 may include components suitable for exposing a target layer of muscle tissue by removing epithelial cells and/or other surface cells.

The receiver preparation sub-system 202 may include components to remove undesired cells or cell layers from a receiver material 204 using any technique known in the art including, but not limited to, laser ablation, mechanical separation, or chemical techniques.

In some embodiments, the system 200 includes a bioprinting sub-system 206 suitable for printing donor material 208 including one or more cells of a desired type onto a receiver material 204. The bioprinting sub-system 206 may include any combination of components suitable for printing (e.g., depositing) donor material 208 onto the receiver material 204. For example, the bioprinting sub-system 206 may include components suitable for bioprinting using a laser-induced forward transfer (LIFT) technique, an extrusion technique, or an inkjet technique. Further, the components of the bioprinting sub-system 206 may be arranged to print the donor material 208 onto the receiver material 204 while the receiver material 204 is connected to the patient (e.g., during surgery).

As used herein, “bioprinting” means utilizing three-dimensional, precise deposition of biological material (e.g., cells, cell solutions, cell-containing gels, cell suspensions, cell concentrations, multicellular aggregates, multicellular bodies, proteins, nucleic acids, extracellular material, intracellular material, or a scaffolding material, etc.) via methodology that is compatible with an automated, computer-aided, three-dimensional prototyping device (e.g., a bioprinter).

Referring now to FIG. 1A, various steps of the method 100 are described in greater detail, in accordance with one or more embodiments of the present disclosure. In some embodiments, the method 100 includes a step 102 of generating donor material 208. The donor material 208 may include any material that is to be deposited onto the receiver material 204, including but not limited to a cell, a tissue, a protein, a nucleic acid, an extracellular material, an intracellular material, or a scaffolding material. For example, the donor material 208 may be a urothelial cell, a fibroblast, a mesenchymal cell, an adipocyte, a keratinocyte (e.g., esophageal keratinocytes), a chondrocyte an immune cell, a muscle cell, a nerve cell, an insulinogenic cell, or a stem cell. For instance, the donor material 208 may include urothelial cells that were harvested from bladder tissue or bladder-like tissues (e.g., urothelial cells scraped from a portion of a bladder, ureter, urethra, or renal pelvis). In another instance, the cell may be an insulinogenic B. In another instance, the donor material 208 may include an intestinal epithelial cell.

It should be understood that the donor material 208 may be derived from the recipient of the tissue transplant (e.g., an autologous transplant), or from another party (e.g., a heterologous transplant). It should also be understood that the cellular portions of the donor material may be derived, differentiated, or otherwise isolated from primary or non-primary sources. For example, the donor material 208 may be a progenitor cell (i.e., a cell having non-proliferative or low-proliferative qualities). In another example, the donor material 208 may be a stem cell, having high proliferative and/or differentiating capacity. For instance, the donor material 208 may comprise unipotent stem cells capable of producing urothelial cells. In particular, unipotent urothelial cells may be isolated from a bladder, a ureter, a urethra, or a renal pelvis, expanded in vitro, then transferred to the receiver material 204.

In another example, the donor material 208 may include multipotent stem cells, capable of differentiating into more than one cell type. For instance, the donor material 208 may include endoderm stem cells, or stem cells arising from an endoderm lineage. In particular, multipotent mesenchymal cells (e.g., derived from hemopoietic or adipose tissue) may be expanded and differentiated towards a urothelial fate. The resultant urothelial cells may then be transferred to the receiver material. In another example, the donor material 208 may include mesoderm stem cells or stem cells arising from a mesoderm lineage. In another example, the donor material 208 may include ectoderm stem cells or stem cells arising from an ectoderm lineage. In other words, stem cells may arise from a mesodermal, endodermal, and or ectodermal origin, and may come from a common host (e.g., the host that provides intestinal tissue for cystoplasty).

In another example, donor material 208 may include pluripotent stem cells capable of producing endodermic, mesodermic, or ectodermic lineages of cells. For instance, cells from the patient may be induced to become induced pluripotent stem cells (iPS). The resultant iPS cells are then expanded and differentiated into urothelial cells, which may then be transferred to the receiver material 204.

In some embodiments, the step 102 includes the isolation of primary urothelial cells (pUCs). For example, primary urothelial cells may be isolated from biopsies of any urothelial tissue, i.e., renal pelvis, ureters, bladder, and proximal urethra. The most accessible among those is the bladder. For example, prior to cystectomy, patients may undergo histological evaluation of transurethral biopsies in order to accurately assess tumor stage. In this application, additional biopsies coming from unaffected areas of the bladder may be removed for pUC isolation. If this is impractical, proximal urethra may also be used. Prior to processing for pUC isolation, mirror image halves of each biopsy may be assessed by a certified pathologist to confirm/verify that the biopsy is tumor-free or cancer-free.

In some embodiments, the step 102 includes expansion of isolated donor material 208. This expansion may in some cases be performed in vitro. For example, isolated donor material 208 and/or a tissue from a biopsy that includes donor material 208 may undergo tissue dissociation in a culture medium. As an illustration, isolated donor material 208 and/or a tissue from a biopsy that includes donor material 208 is submerged in culture media. The volume and concentration of dissociation enzymes as well as the duration of the enzymatic treatment may be adjusted depending on the biopsy weight. Further, the conditions of the expansion may also be adjusted to promote expansion. As an illustration, the sample may be, but is not required to be, submerged for a range of approximately 6-12 hours at any suitable temperature (e.g., ambient temperature, in a cooled (e.g., iced) environment, or the like.

In another example of a method for cell expansion, cells may be isolated from biopsies (e.g., healthy biopsies) using keratinocyte serum free media (KSFM) supplemented with serum or a serum replacement. The serum or serum replacement may be of any type including, but not limited to, fetal bovine serum, fetal calf serum, calf serum, human serum, human platelet lysate, and bovine pituitary extract. The supplemented serum may be used at any percentage or range of percentages. For example, the serum may be used in a range between 0.5% to 20% of the total media volume. In another example, the serum may be used at substantially 5% of the total media volume. The media may also include one or more factors including, but not limited to, human epidermal growth factor (hEGF), bovine fibroblast growth factor (FGF) and cholera toxin.

The methods for cell expansion further include the growth of cells within a vessel. The vessel may include any type of vessel capable of supporting cell growth including but not limited to plastic tissue culture vessels (e.g., plates and flasks). The vessels may be coated on a growth area within the vessel with any type of cell-support coating (e.g., chemical, matrix, or cell coating) including, but not limited to, an inactivated-cell feeder coating (e.g., using primary fibroblasts), extracellular matrix (ECM), basement membrane extracts (BME), and Matrigel™. The concentration of the coating material (e.g., concentration of the protein) subjected to the growth area may range from 0.1 to 10 mg/ml. For example, the concentration of the coating material may be 1 mg/ml. The time for coating or passivating the growth area with the coating material may be in a range of 5 minutes to 24 hours. For example, the time for coating or passivating the growth area with the coating material may range from 10 minutes to 30 minutes.

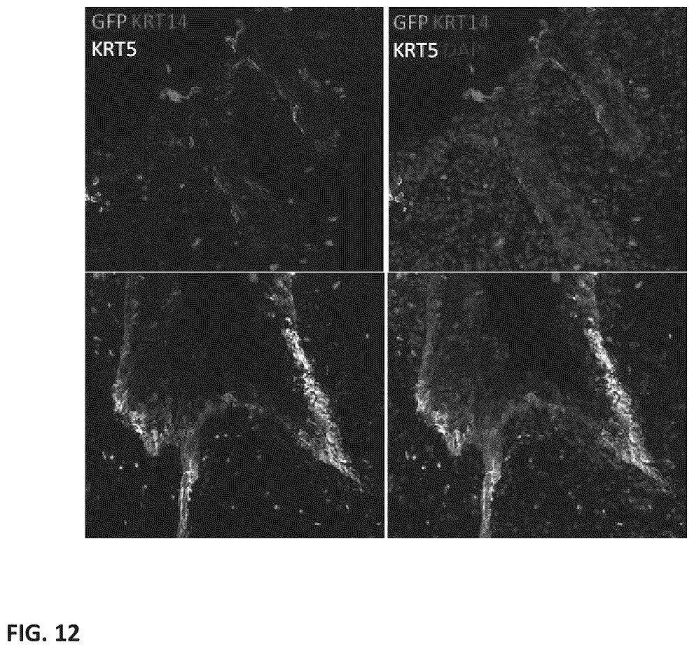

It is contemplated herein that urothelial cells from the bladder or urethral biopsies can be expanded for several passages using appropriate culture media. Cells of the donor material 208 may grow in 2D monolayer or in a bioreactor (e.g., spinner flasks) and may be passaged through enzymatic and mechanical dissociation. Before they reach confluence, cells are passaged 1:3-1:4. Some early passage cells (P1 and P2 cells) may be cryopreserved in appropriate cryopreservative media in liquid nitrogen (vapor phase). Also, early and late passage cells may be monitored for their ploidy status (karyotype analysis on mitotic spreads). In case the surgery is canceled and postponed for later than a week, frozen cells are thawed and expanded. The cells can be cultured under GMP-compliant conditions in typical cultures or bioreactors. Under these conditions, cells maintain the characteristics of the basal layer of the urothelium, where stem cells reside. When grown on 3D cell matrices, cells grow as 3D organoids (spheres), without losing their cell identity. Organoid forming efficiency measurement provides an initial assessment of the quality of the cells and their functionality. In all cases, monitoring of cellular identity will be applied to several samples prior to placation. Cells grown on culture dishes will be investigated through Flow Cytometry and organoids through Immunofluorescence, as shown in FIG. 12. Only positive cultures of cells against basal urothelial markers like keratin 5, α6 Integrin (70%) and negative for superficial differentiation ones such as keratin 20 (<10%) will be allowed for clinical application. Similarly, only fully differentiated organoid cultures with the abovementioned basal markers on the periphery and superficial on their core (>70%) will be utilized in the clinic.

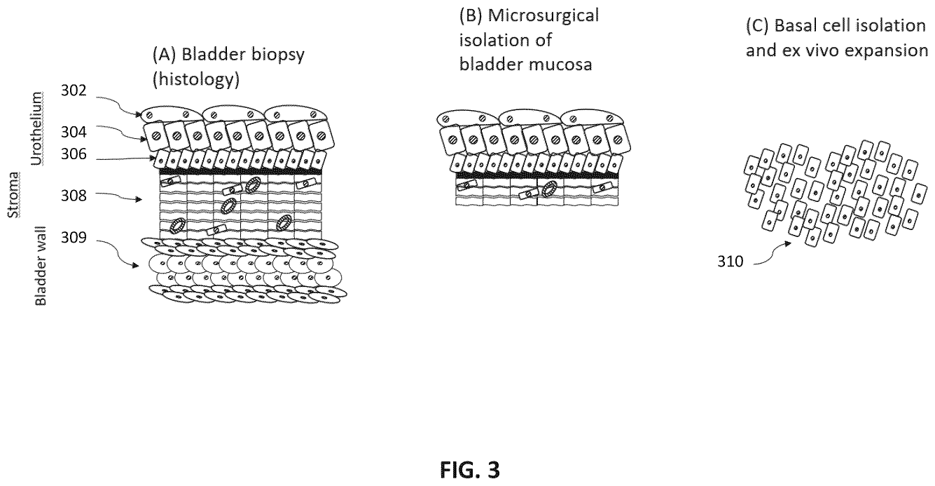

FIG. 3 is a simplified diagram illustrating the isolation and expansion of donor material 208 in step 102, in accordance with one or more embodiments of the present disclosure. Panel (A) is a schematic representation of bladder histology, in accordance with one or more embodiments of the present disclosure. For simplicity, some structures are not depicted (for instance, serosa, vasculature, or nerves). The urothelium includes three distinct cell types in layers of different thicknesses among species. However, umbrella cells 302 are always on the top facing the lumen, intermediate cells 304 in the middle layer, and basal cells 306 comprise the bottom layer of the urothelium. Beneath the urothelium lies lamina propria (e.g., stromal tissue 308) which harbors several cell types (including fibroblasts and immune cells) intense extracellular mesh (fibrous connective tissue including several types of collagen) and is where bladder vasculature concludes). The outermost bladder tissue may consist of smooth muscle cells (detrusor muscle), which is also called bladder wall 309.

Panel (B) is a schematic representation of the isolation of urothelium from a bladder biopsy. This process may be conducted microsurgically under aseptic conditions. Panel (C) Following the pUC isolation protocol, the urothelial cells 310 that eventually attach to the culture dish and proliferate are only the basal cells 306. The donor material 208 may then include these urothelial cells 310.

Referring again to FIG. 1A, in some embodiments, the method 100 includes a step 104 of removing a portion of a receiver material 204 including biological tissue to expose a target layer of the receiver material 204 for placement of the donor material 208. The step 104 may be performed using any technique known in the art including, but not limited to, laser ablation, mechanical separation, or chemical techniques.

The material removed in step 104 may include any cells, cellular material, extracellular tissue (e.g., extracellular matrix), tissue, organ, or portion of an organ. For example, the material removed may include crypt cells (i.e., intestinal epithelial cells) removed from intestinal tissue. In another example, the material removed may include hyperplasias (e.g., small masses of proliferating cells). In another example, the material removed may include one or more mucosal layers.

As used herein, “organ” refers to a collection of tissues joined into structural unit to serve a common function. Examples of organs include, but are not limited to, skin, sweat glands, sebaceous glands, mammary glands, bone, brain, hypothalamus, pituitary gland, pineal body, heart, blood vessels, larynx, trachea, bronchus, lung, lymphatic vessel, salivary glands, mucous glands, esophagus, stomach, gallbladder, liver, pancreas, small intestine, large intestine, colon, urethra, kidney, adrenal gland, conduit, ureter, bladder, fallopian tube, uterus, ovaries, testes, prostate, thyroid, parathyroid, meibomian gland, parotid gland, tonsil, adenoid, thymus, and spleen.

As used herein, “stem cell” means a cell that exhibits potency and self-renewal. Stem cells include, but are not limited to, totipotent cells, pluripotent cells, multipotent cells, oligopotent cells, unipotent cells, and progenitor cells. Stem cells may be embryonic stem cells, peri-natal stem cells, adult stem cells, amniotic stem cells, and induced pluripotent stem cells.

As used herein, “tissue” means an aggregate of cells. Examples of tissues include, but are not limited to, connective tissue (e.g., areolar connective tissue, dense connective tissue, elastic tissue, reticular connective tissue, and adipose tissue), muscle tissue (e.g., skeletal muscle, smooth muscle and cardiac muscle), genitourinary tissue, gastrointestinal tissue, pulmonary tissue, bone tissue, nervous tissue, and epithelial tissue (e.g., simple epithelium and stratified epithelium), endoderm-derived tissue, mesoderm-derived tissue, and ectoderm-derived tissue. As used herein, “dysmorphism” is the condition of having an abnormally shaped body part, organ, or tissue.

In some embodiments, the method 100 includes a step 106 of printing the donor material 208 onto the target layer of the receiver material 204. The step 106 may be performed using any technique known in the art including, but not limited to, a LIFT technique, an extrusion technique, or an inkjet technique.

The receiver material 204 may include any tissue, organ, organoid, graft, or other material that may be a substrate for tissue engineering and/or transplantation. For example, the receiver material 204 may be a tissue excised from a patient (e.g., a human and/or animal patient) that will be transplanted back into the patient after modification by the system 200. For instance, the receiver material 204 may be intestinal tissue that has been excised from the patient and modified by the system 200, then repurposed as a bladder wall component in order to increase the size and urine holding capacity of the bladder, a technique known as augmentation cystoplasty. In another instance, insulinogenic B-cells may be similarly printed on intestinal smooth muscle, or onto any other splanchnic tissue. In still another instance, colonic cells may be similarly printed on intestinal smooth muscle for large intestine regeneration. Similar techniques may also be used for ureter and urethra engineering. The receiver material 204 may be any source of muscle tissue (e.g., smooth muscle tissue) with or without stromal tissue). The receiver material 204 may also include other biological tissues including but not limited to smooth muscle tissue, skeletal muscle tissue, blood vessels, skin, bone, connective tissue (e.g., facia), epithelial tissue, and nervous tissue. For example, the receiver material 204 may include any section of the gastrointestinal tract (e.g., stomach), the diaphragm, the uterus, or the fallopian tubes. In another example, the receiver material 204 may include bladder tissue, cartilaginous tissue, or esophageal tissue.

It is contemplated herein that the step 104 and/or the step 106 may be performed ex-vivo or in-vivo. In either case, ex-vivo applications may be performed on a receiver material 204 that is not connected to a patient prior to transplantation, whereas in-vivo applications may be performed on a receiver material 204 that is at least partially connected to the patient (e.g. during a surgical procedure).

In some embodiments, the method 100 includes a step 108 of applying polymerized acellular glucosaminoglycan (GAG) or platelet-rich plasma (PRP) to the receiver material 204 after printing the donor material 208 onto the target layer of the receiver material 204. This step 108 may be performed using any technique known in the art such as, but not limited to, a LIFT technique, an extrusion technique, or an inkjet technique. Further, the step 108 may be performed using the same technique as for printing the donor material 208 in step 106 or by a different technique. In some embodiments, the step 104 and/or the step 106 may be performed during a surgical procedure.

As a non-limiting illustration, the step 104 and/or the step 106 performed during a bladder augmentation or orthotopic neobladder procedure are described, in accordance with one or more embodiments of the present disclosure. For the bladder, the surgical procedure may include a typical cystectomy/cystoplasty operation with a small modification: the intestinal part (e.g., a receiver material 204) is deprived of its epithelium (e.g., in step 104) and pUCs are bioprinted to replace it (e.g., in step 106). A portion (typically ˜40 cm) of the intestine (small or large) may be removed and de-tubularized at the antimesenteric side by cutting longitudinally at the antimesenteric side (e.g., with the surgeon holding one side and the assistant the other at 5 cm steps).

After washing the exposed luminal part with warm saline and gauges, the surgeon may separate the mucosal part of the receiver material 204 from the remaining tissue (mainly muscularis externae and part of the lamina propria) using any suitable technique (e.g., step 104). For example, step 104 may be implemented using mechanical techniques by holding the muscular part of the receiver material 204 with forceps (e.g., large Debakiy forceps, or the like) and separating the mucosal is separated with additional forceps. In some instances, once the first 2-3 cm of the intestinal mucosa is separated the muscular tissue can be held with hands as the manual mechanical denudation proceeds until complete separation throughout the intestinal segment is achieved. At this point, the receiver material 204 is then ready to be bioprinted (e.g., in step 106) using any suitable technique.

In some embodiments, one or more additional steps are performed to arrange the receiver material 204 into a desired shape for transplantation. For example, the receiver material 204 may be bioprinted directly (long stretch) or after being reshaped in a typical “U” or “S” shape with continuous sutures.

In some embodiments, the bioprinting step (step 106) is performed in vivo or ex vivo. For example, the receiver material 204 may be printed onto the organ before transplantation into the patient (e.g., ex vivo). In another example, the receiver material 204 may be printed onto the organ after transplantation into the patient (e.g., in vivo). It is contemplated herein that the receiver material 204 may generally need to remain in close proximity to its original position in the patient, where the ability to move and/or manipulate the receiver material 204 may depend on the particular procedure.

As an illustration in the case of a receiver material 204 including denuded intestine, the denuded intestine needs to be spread flat and immobilized thereon with sutures, clips or musket forceps in opposing sites in order to keep the tissue stretched and flat, with extreme care in order to avoid damaging the tissue. The denuded intestine is compliant to stretching but not in displacement from its original position by more than a few centimeters. The printing of pUCs (e.g., step 106) may precede tissue reshaping (e.g., shaping) or be conducted just after and the cystectomy/cystoplasty procedure resumes accordingly. This method can be conducted with only the printing of autologous pUCs. However, it can also proceed with sequential printing of autologous pUCs and GAG (e.g., in step 108), or with ex vivo pUC:GAG preparations.

As another non-limiting illustration, the step 104 and/or the step 106 performed during a urethroplasty are described, in accordance with one or more embodiments of the present disclosure. For urethra, the surgery is a typical urethroplasty for large strictures (>2-3 cm long) but without the use of autologous graft tissue (like oral mucosa tissue or genital skin), which is replaced with direct bioprinting (e.g., step 106) of autologous pUCs alone, sequential bioprinting of pUCs and GAG (e.g., step 108) or appropriate ex vivo pUC:GAG composite preparations. The typical urethroplasty process proceeds up to the removal of damaged urethral urothelium. Then the printing of pUCs or sequential printing of pUCS and GAG or the anastomosis of the tubular ex vivo pUC:GAG preparation takes place. Then the typical urethroplasty resumes after grafting of the autologous donor material 208. In some embodiments, the receiver material 204 itself may include GAG.

A glucosaminoglycan (GAG) layer on the luminal side of the umbrella cells may improve the waterproof aspects of the blood-urine barrier and consists mainly of hyaluronic acid (HA) and chondroitin sulfate (CS). CS and HA are commercially available and can be easily modified to become methacrylated or purchased as methacrylated (e.g., MA-HA, MA-CS). MA-HA, MA-CS, or mixtures of MA-HA and MA-CS in 1:4 up to 4:1 ratios can be utilized to form an acellular matrix that can be photopolymerized with UV or green light, depending on the photo-initiator utilized (e.g., Irgacure 2959 or Lithium phenyl-2,4,6-trimethylbenzoylphosphinate-LAP). The polymerization step may last for a few seconds up to several minutes depending on the desired thickness and stiffness of the end product. GAG can be photopolymerized in any given or developed mold of desired size/shape. Both HA and CS are approved for clinical applications. GAG scaffolds (e.g., receiver material 204) can be generated under GLP compliance and aseptic conditions. UCs (e.g., donor material 208) may be printed (e.g., in step 106) on solidified GAG, ex-vivo, using LIFT or inkjet or extrusion process. The pUC:GAG composite may then be directly sutured on denuded intestinal tissue, during the in-vivo protocols, or after a few days of ex-vivo proliferation and differentiation towards stratified urothelium.

To proliferate and differentiate the UCs on the UC:GAG composite, the composite may be submerged or placed on culture medium (air-liquid interface culture) in appropriate containers and will be incubated in a typical cell culture incubator (37 degrees Celsius, 5% CO₂). The medium (same as UC growth medium) will be supplemented with 2-6M CaCl₂ in order to induce differentiation of UCs. The differentiation medium may be replenished every other day and the growth will be monitored with microscopy. UC:GAG composite samples can be excised and processed histopathologically, generating formalin-fixed paraffin-embedded or OCT-embedded sections for histological and histochemical staining. Such stainings include, but are not limited to, Haematoxylin-Eosin, Masson's Trichrome staining, immunohistochemical and immunofluorescent staining against antibodies relevant to bladder physiology (e.g. keratins). Successful urothelial differentiation would result in three distinct layers of cells with the basal cell layer being a cell layer expressing basal keratins (like keratin 5) and the uppermost layer being positive for superficial markers (such as keratin 20 and AUM).

For the replacement of damaged urethra, the same principles apply as above, but the GAG scaffold will be molded in tubular form. The size of the mold is personalized to the patient. The length will be determined by the stricture size plus 0.5 cm. The thickness will be 1-3 mm and the diameter will be determined by the size of the catheter that will be utilized in the surgery plus 0.5 mm.

Another method 150 for tissue reconstruction is illustrated in FIG. 1B. The method 150 may include one or more steps of method 100, and vice versa. In embodiments, the method 150 includes a step 154 of extracting donor cells from donor tissue of a patient. In embodiments, the method 150 includes a step 158 of generating donor material 208, which may include the donor cells (e.g., primary donor cells, expanded donor cells, or modified donor cells). In embodiments, the method 150 includes a step 162 of removing (e.g., via ablation, mechanical separation, chemical separation, or enzymatic separation) one or more cell layers from a receiver tissue of the patient to expose a target layer of the receiver tissue while maintaining operation of a vasculature of the receiver tissue. In embodiments, the method 150 includes a step 166 of printing the donor material 208 (e.g., via LIFT, extrusion, or an inkjet process) onto the target layer of the receiver tissue (e.g., receiver material 204). In embodiments, the method 150 includes a step 170 of verifying that the donor material is free of cancer cells (e.g., via histology or other known pathology methods). In embodiments, the method 150 includes a step 174 of grafting the receiver tissue to an additional tissue of the patient while maintaining the operation of a vasculature of the receiver tissue (e.g., the receiver tissue maintains competent blood flow).

Referring now to FIG. 2, various aspects of the system 200 are described in greater detail, in accordance with one or more embodiments of the present disclosure. The receiver preparation sub-system 202 may generally include any components or combination of components suitable for selectively removing undesired cells or cell layers from a receiver material 204. Put another way, the receiver preparation sub-system 202 may generally include any components or combination of components suitable for preparing a receiver material 204 for bioprinting of donor material 208.

In some embodiments, the receiver preparation sub-system 202 includes a laser ablation assembly 210 suitable for selectively ablating the one or more cells. For example, the receiver preparation sub-system 202 may include, but is not limited to, components for laser ablation described in U.S. patent application Ser. No. 17/387,801 filed Jul. 28, 2021, to which the present disclosure claims priority and which is incorporated herein by reference in its entirety. In some embodiments, the receiver preparation sub-system 202 includes components suitable for removing one or more cells using chemical techniques.

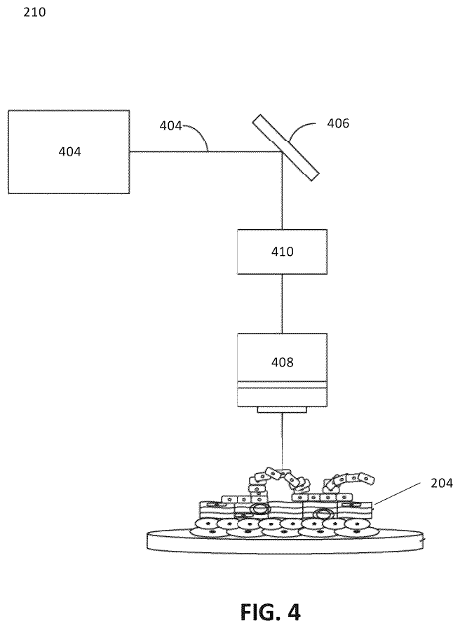

FIG. 4 is a diagram of a laser ablation assembly 210, in accordance with one or more embodiments of the present disclosure. In some embodiments, the laser ablation assembly 210 includes a laser 402 to generate an ablation beam 404 (e.g., an ablation beam). In embodiments, the laser ablation assembly 210 includes one or more optical elements configured to direct the ablation beam 404 to the receiver material 204. The optical elements may be any known in the art including but not limited to mirrors, lenses, and beamsplitters. For example, the optical elements may include one or more reflecting mirrors 406. As another example, the optical elements include a focusing lens 408. The focusing lens 408 may control the size of the ablation beam 404 upon the receiver material 204. The focusing lens 408 may be any type of lens known in the art including but not limited to an achromatic lens. For example, the focusing lens 408 may be a 150 mm achromatic lens. In another example, the focusing lens 408 may be a microscope objective.

The laser 402 may include any laser known in the art used for ablating material including but not limited to a solid-state laser, a gas laser, a dye laser, or a semiconductor laser. For example, the laser 402 may include a diode-pumped solid-state laser. As one illustration, the laser 402 may include a diode-pumped Nd:YAG solid-state laser.

The ablation beam 404 produced by the laser 402 may include any wavelength or wavelength range suitable for selectively removing cells to expose a target layer of a receiver material 204. For example, the laser 402 may produce ablation beam 404 with wavelengths in the visible spectrum (e.g., 380 to 780 nm). As another example, the laser 402 may produce ablation beam 404 with wavelengths in the near-infrared spectrum (e.g., 780 to 2500 nm). For instance, the ablation beam 404 may include a wavelength of approximately 1064 nm.

In some embodiments, the ablation beam 404 produced by the laser 402 may be pulsed. The repetition rate of the ablation beam 404 may be any repetition rate or range of repetition rates known in the art. For example, the laser 402 may produce an ablation beam 404 with a repetition rate ranging from 100 Hz to 10 KHz. In another example, the laser 402 may produce an ablation beam 404 with a repetition rate ranging from 300 Hz to 3 kHz. In another example, the laser 402 may produce an ablation beam 404 with a repetition rate of approximately 1 kHz. In another example, the laser 402 may produce a singular pulse.

In some embodiments, the laser 402 produces a pulsed ablation beam 404 with a selected pulse length or range of pulse lengths. The ablation beam 404 may have any pulse length suitable for selectively removing cells to expose a target layer of a receiver material 204 such as, but not limited to, a pulse length on the order of femtoseconds (fs) to nanoseconds (ns). For example, the length of the pulse of the ablation beam 404 may range from 60 ps to 6 ns. In another example, the length of the pulse of the ablation beam 404 may range from 100 ps to 30 ns. In another example, the length of the pulse of the ablation beam 404 may be approximately 600 ps.

In some embodiments, the laser 402 produces an ablation beam 404 with a specific fluence or range of fluences (e.g., optical energy delivered per unit area). The fluence of the ablation beam 404 may be any range or value known in the art. For example, the fluence of the ablation beam 404 may range from 10 mJ/cm² to 10 J/cm². In another example, the fluence of the ablation beam 404 may range from 100 mJ/cm² to 1 J/cm². In another example, the fluence of the ablation beam 404 may range from 100 mJ/cm² to 500 mJ/cm². In still another example, the fluence of the ablation beam 404 may range from 300 mJ/cm² to 800 mJ/cm².

The ablation beam 404 may be focused to any spot size on the receiver material 204 by the focusing lens 408. For example, the spot size may range from 10 um to 1 mm in diameter. In another example, the spot size may range from 30 um to 300 um in diameter. In another example, the ablation/removal spot may be approximately 100 um

In embodiments, the laser ablation assembly 210 includes an optical attenuator 410 configured to modify the ablation beam 404. The optical attenuator 410 may be any optical attenuator known in the art including but not limited to a fixed attenuator, a loopback attenuator, an adjustable attenuator, or a variable optical test attenuator. For example, the optical attenuator 410 may be a fixed attenuator plate.

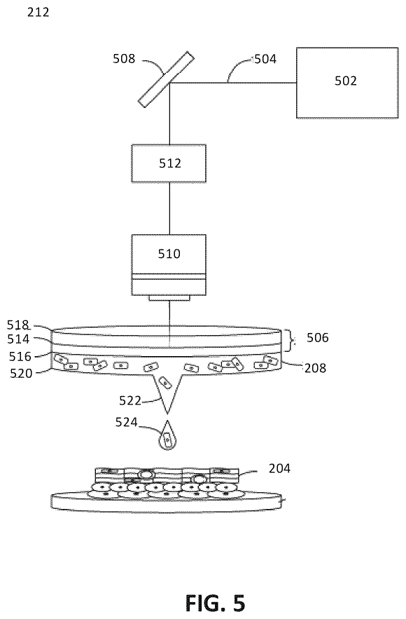

Referring again to FIG. 2, the bioprinting sub-system 206 may include any components or combinations of components suitable for printing donor material 208 onto a receiver material 204. In some embodiments, the bioprinting sub-system 206 includes a LIFT assembly 212. For example, the step 106 may include, but is not limited to, components for LIFT printing of donor material 208 described in U.S. patent application Ser. No. 17/387,801 filed Jul. 28, 2021, to which the present disclosure claims priority and which is incorporated herein by reference in its entirety. FIG. 5 is a schematic diagram of a LIFT assembly 212, in accordance with one or more embodiments of the present disclosure.

In some embodiments, the LIFT assembly 212 includes a laser 502 to generate a LIFT beam 504. In a general sense, the LIFT beam 504 may have any suitable spatial or temporal properties. In this way, the descriptions of the ablation beam 404 above may be generally extended to apply to the LIFT beam 504. Further, it is contemplated herein that the laser 502 may be the same as the laser 402 described with respect to a laser ablation assembly 210 or may be different.

In some embodiments, the LIFT assembly 212 includes one or more optical elements configured to direct the LIFT beam 504 to a donor substrate 506. The optical elements may be any known in the art including but not limited to mirrors, lenses, and beamsplitters. For example, the optical element may include the one or more reflecting mirrors 508. In another example, the optical element may include a focusing lens 510 to focus the LIFT beam 504 onto the donor substrate 506. For instance, the focusing lens 510 may be a 75 mm achromatic lens. In some embodiments, the LIFT assembly 212 includes an optical attenuator 512 to control the intensity (or fluence) of the LIFT beam 504 on the donor substrate 506, which may be fixed or tunable.

In preparation for transfer of the donor material 208, the donor substrate 506 contains, holds, or is otherwise coupled or loaded with, the donor material 208. The donor substrate 506 aids in the transfer of the donor material 208 to the receiver material 204. In some embodiments, the donor substrate 506 comprises a front surface 514. The front surface 514 faces the receiver material 204 and may be, but is not required to be, coated with a laser absorbing layer 516 (e.g., a dynamic release layer), that absorbs energy from the LIFT beam 504 and facilitates precise transfer of the donor material 208 to the receiver material 204. The donor substrate 506 further includes a back surface 518 that initially receives the LIFT beam 504.

In some embodiments, the donor material 208 includes a suspension 520 including cells of the desired type, which is coated over the back surface 518 of the donor substrate 506 (e.g., on a laser absorbing layer 516, if present). As an illustration, once the LIFT beam 504 reaches the laser absorbing layer 516, localized heating at the laser absorbing layer 516 and/or the suspension 520 create a high-pressure vapor bubble 522 within the localized area of the suspension 520. The expansion of the vapor bubble 522 then drives the ejection of a droplet 524 of the suspension 520 towards the receiver material 204 for deposition (e.g., printing) of the associated cells.

In some embodiments, the LIFT assembly 212 includes a donor supply sub-system 214 to provide a supply of donor material 208 for bioprinting. It is contemplated herein that a central obstacle in all bioprinting technologies is the speed of the printing and the speed of the supply of the donor cells donor.

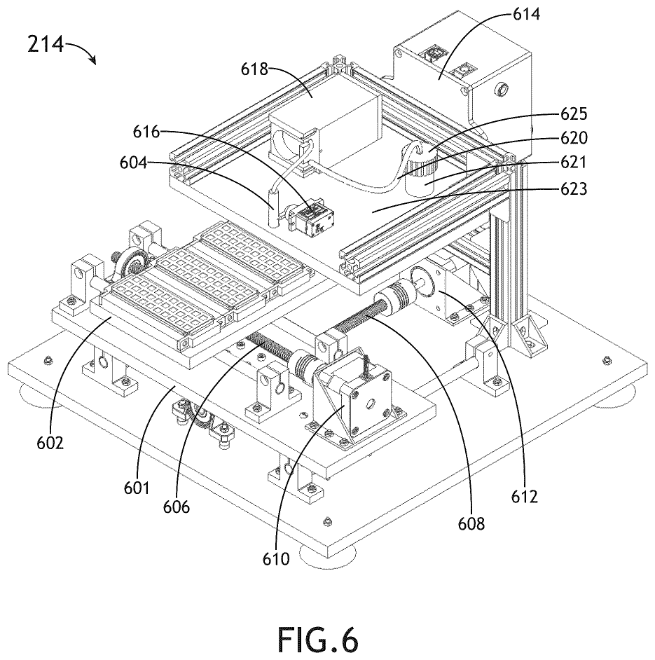

FIG. 6 is a perspective view of a schematic of a donor supply sub-system 214, in accordance with one or more embodiments of the present disclosure. The donor supply sub-system 214 supplies donor material (e.g., cells) to the bioprinter (e.g., bioprinting sub-system 206).

In some embodiments, the donor supply sub-system 214 includes a robotic platform 601 (e.g., an automated stage) with two horizontal degrees of freedom via X-axis guides and Y-axis guides, and an dispenser subsystem that provides an outlet for the donor material 208 that includes an automatic material feeding head 604 with one degree of freedom and a pump 618. In some embodiments, the robotic platform 601 described includes lead screws 606,608 (e.g., X-axis screw shafts and Y-axis screw shafts) actuated by stepper motors 610,612 (e.g., an X-axis motor and a Y-axis motor), which may be controlled by one or more controllers 614 communicatively coupled to the robotic platform 601 and/or the dispenser subsystem. The one or more controllers 614 may include any type of controller known in the art including, but not limited to, a microcontroller. The one or more controllers 614 may include one or more processors, and a memory. For example, the one or more processors may be configured to execute a set of program instructions stored in a memory (e.g., the program instruction are configured to cause the one or more processors to execute the program instructions). The set of program instructions may then instruct the one or more processors to operate the pump 618, which transports donor material 208 from the reservoir 621 through the material feeding head 604. The set of program instructions may also instruct the one or more processors to operate the robotic platform 601 to move the donor frame 602 in coordination with an operation of the pump 608, wherein donor material 208 is transported to a designated area via a movement of the donor frame 602 and the operation of the pump 618.

The material feeding head 604 may be actuated by a servo motor 616 and controlled with the same controller 614 or a dedicated one. The material feeding head 604 may be provided material, through the pump 618 (e.g., a micro peristaltic pump, or the like) with high accuracy and may be capable of delivering reliably small quantities of donor material 208 from a tube 620 supplied from a reservoir 621 at an inlet 625 (e.g., the reservoir 621 secured at a reservoir stage area 623).

During operation, the robotic platform 601 may position a specific part of a plate or dish underneath the material feeding head 604. The material feeding head 604 may then be lowered within a specific distance from the surface of the plate or dish and donor material 208 is pumped through the pump 618 and deposited on the surface of the donor substrate 506. The material feeding head 604 may be further lowered to come into contact with the surface of the donor substrate 506. The robotic platform 601 may then move in a specific pattern, for example a rectangular spire, that helps disperse the donor material 208 uniformly within the area of the donor substrate 506 that has been selected. The process is repeated any number of times.

Referring again to FIG. 2, in some embodiments, the system 200 includes a tissue position assembly 220 to secure and/or position the receiver material 204 (e.g., during any of step 104, step 106, or step 108 through any suitable technique. For example, in the case of LIFT printing, an appropriate receiver module can be designed in order to aid in securing or gripping of a flat and stretched tissue without interfering with mesenteric tissue (e.g., keeping vascularization unperturbed).

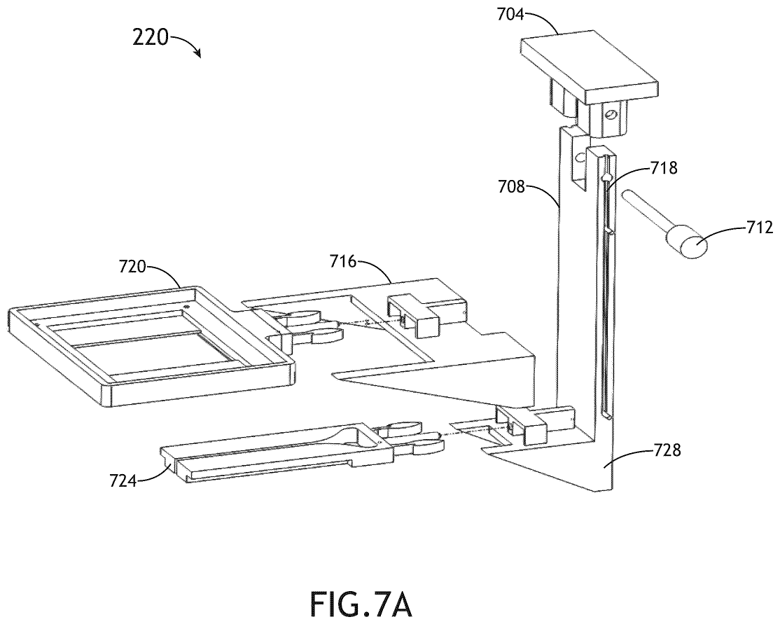

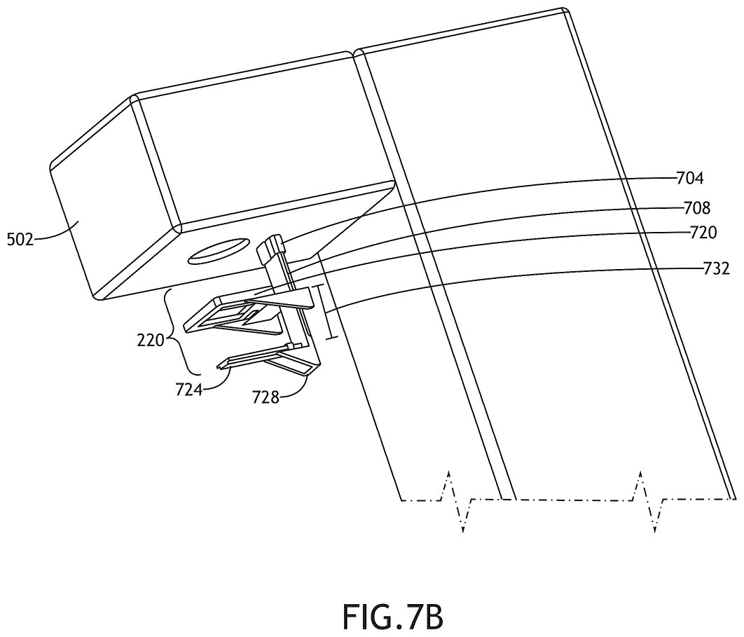

In embodiments, the tissue position assembly 220 includes the tissue position assembly 220 as described in FIGS. 7A-7B. The tissue position assembly 220 includes a base 704, and a main rail 708. The base 704 and main rail 708 may be coupled via connecting mechanism 712 (e.g., such as a dowel that is thrust through aligned holes in both the base 704 and the main rail 708). The base 704 may also have one or more slots or extrusions that slide-fits with one or more pegs or extrusions located at the top of the main rail 708. In embodiments, the tissue-securing assembly includes a carriage 716 slidably coupled to the main rail 708. The carriage 716 may be configured to slide linearly on the main rail 708 through a rail slot 718 and can hold position (e.g., be positionable) at different places along the main rail 708. For example, the carriage may be configured to hold position on the main rail 708 through 45-degree turns at the beginning and end of the sliding route. The carriage 716 is configured to couple to a donor plate 720 (e.g., a type of donor substrate 506). The donor plate 720 is configured to hold cells (e.g., donor material 208) for transfer via LIFT.

The tissue position assembly 220 further includes a receiver plate 724 configured to couple to the rail end 728 of the main rail 708 and to support and secure receiver material 204. The receiver plate 724 couples to the rail end 728 via a connecting mechanism such as a screw, pin, buckle, or interference (e.g., snap or friction) mechanism. The donor plate 720 couples to the carriage 716 via a connecting mechanism such as a screw, pin, buckle, or interference (e.g., snap or friction) mechanism.

The integration of the tissue position assembly 220 onto the LIFT assembly 212 (e.g., with the laser 502) is shown in FIG. 7B. The base 704 is shown coupled to the housing of the laser 502, with the main rail 708 coupled to the base. The donor plate 720 is stably positioned along the main rail 708 via the carriage 716. The receiver plate 724 is coupled to the rail end 728 of the main rail 708. The sliding of the donor plate 720 and carriage 716 along the main rail 708 allows the operator to adjust an operating distance of the tissue securing assembly (e.g., for securing different sizes of receiver material 204 or receiver material containers, or for adjusting the distance that cells will travel when being printed from the donor plate 720 onto the receiver plate 724.

In embodiments, one or more components of the tissue position assembly 220 are disposable. For example, the donor plate 720 and/or the receiver plate 724 may be constructed of disposable material (e.g., medical-grade plastics) and discarded after one use. In embodiments, one or more components of the tissue position assembly 220 are sterilizable, such as the main rail 708, the base 704, and/or the carriage 716. In embodiments, one or more components of the tissue position assembly 220 (e.g., the carriage 716) are components made of rigid material with anticorrosion properties, such as aluminum or stainless steel. In embodiments, one or more components of the tissue position assembly 220 (e.g., the donor plate 720 and/or the receiver plate 724) are made of plastic materials that are suitable for contact with living tissue, such as material including, but not limited to, polyurethane, polycarbonate, polyethylene, and polypropylene.

In embodiments, tissue position assembly 220 includes a housing configured to secure one or more lenses configured to focus laser light from the laser 502 onto the donor plate 720 (e.g., forcing material on the donor plate 720 onto the receiver material 204. The housing may be coupled or otherwise connected to the main rail 708. The housing may also include one or more windows transparent to laser light for coupling the laser light at least one of into or out of the housing.

In embodiments, a component of the tissue reconstruction system 200, such as the tissue position assembly 220 includes a detector configured to sense or determine a thickness of the tissue secured to the receiver plate 724 (e.g., in a direction of the donor plate 720). For example, the detector (e.g., a laser-, radar-, lidar-, or sonar-based distance detector) may be configured to determine a separation distance between the donor plate 720 and the tissue secured to the replaceable receiver assembly is adjusted based on the thickness of the tissue. In embodiments, the detector is an imaging system 730 (e.g., based on optics, radar, or lidar) that provides a working distance (e.g., a fixed working distance) that is secured on a translation stage, such as the donor plate 720. For example, the detector may determine the thickness of the tissue based on images generated at a different position of the imaging system 730 (e.g., such as on the translation stage), providing an in-focus image of the tissue.

In embodiments, a composition for use in the treatment of organ damage or organ dysmorphism is disclosed. For example, the composition may be used for bladder augmentation, as well as other organ systems described herein. In embodiments, the composition includes donor material 208 (e.g., cells or other donor material 208 suitable for transfer from a donor substrate 506 to receiver material), media (e.g., media suitable for transfer of the donor material from the donor substrate to the receiver material), and componentry as described herein. For example, the composition may include primary cells, cultured cells, stem cells (e.g., pluripotent cells), differentiated cells, and/or undifferentiated cells. For instance, the composition may include urothelial cells (e.g., primary urothelial cells). The composition may include organoids (e.g., organoids containing primary urothelial cells). In embodiments, the composition may include media (e.g., cell media) and/or media components including but not limited to keratinocyte serum free media (KSFM), GAG, HA, and PRP.

In embodiments, the composition may include any substrate used to contain the cells including, but not limited to, the donor substrate 506, and/or donor plate. For example, the composition may include a donor substrate 506 that is loaded with donor material (e.g., cells) and/or media. In another example, the donor substrate 506 may be configured to include a glass substrate layer and a metallic film layer.

In embodiments, an augmented bladder is disclosed. The augmented bladder is augmented via LIFT and other methods and materials described herein. The augmented bladder may be formed and/or augmented ex vivo. For example, the bladder may be excised from the subject, and the augmentation procedure is performed outside of the body.

In embodiments, the augmented bladder includes a first portion of a bladder of a patient (e.g., a main body of a bladder of a subject pre-surgery/augmentation), and a graft formed from intestinal tissue of the patient. The graft may include undisturbed vasculature, with competent blood circulation.

In embodiments, the intestinal tissue includes a layer of donor material 208. The donor material 208 may include cells, such as urothelial cells, iPS cells, or any other cell type as described herein. The donor material 208 is printed into the intestinal tissue via a method that includes a step of extracting donor material 208 (e.g., cells) from donor tissue of the patient (e.g., through cell scraping, chemical dissociation, or other means described herein). The method may also include generating the donor material 208 including the donor cells. Generating the donor material 208 may include common cell expansion methods. The method may also include a step of removing one or more cell layers from the intestinal tissue of the patient (e.g., the epithelium or tubular structures) to expose a target layer of the intestinal tissue while maintaining operation of a vasculature of the intestinal tissue. The method may also include a step of printing the donor material onto the target layer of the intestinal tissue (e.g., via LIFT, inkjet, or an extrusion process). In embodiments, the method for producing the augmented bladder may include a step of placing additional material, such as platelet-rich plasma (PRP) or polymerized acellular glucosaminoglycan (GAG), on the target layer of the intestinal tissue prior to printing the donor material onto the target layer of the intestinal tissue.

In embodiments, the one or more cell layers of the bladder are removed to expose the target layer of the receiver material 204 without disrupting vasculature of the receiver material 204. Techniques to remove the cell layers include but are not limited to laser ablation, mechanical separation, chemical separation, and enzymatic separation.

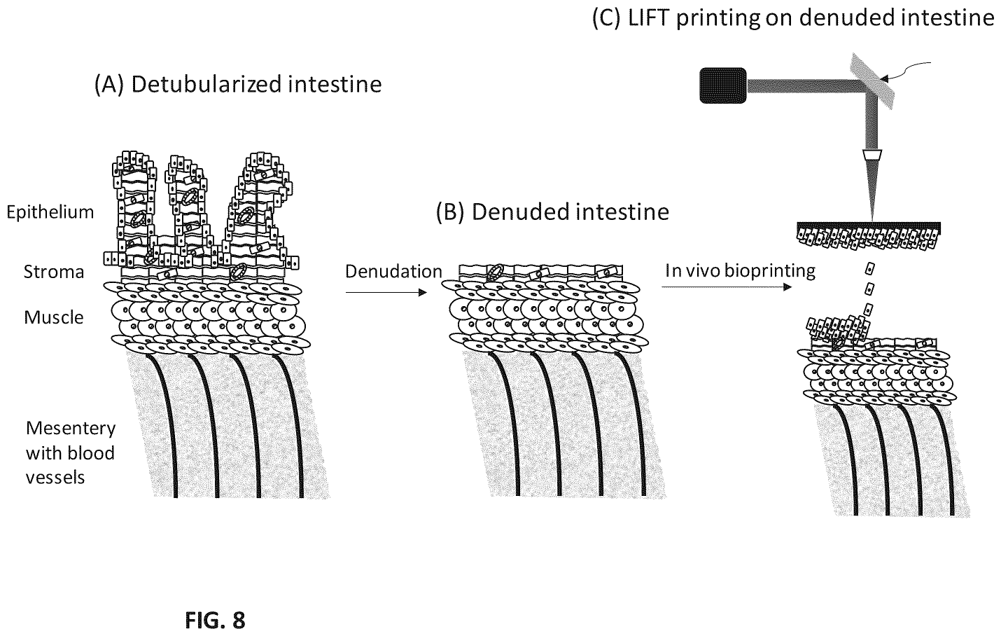

Referring now to FIGS. 8-12, various non-limiting examples of tissue reconstruction using the systems and methods disclosed herein are described in greater detail.

### Example 1. Isolation of pUCs and Stromal Cells (Human-Porcine) (e.g., as Part of Step 102)

Porcine primary urothelial cells (pUCs) were isolated from bladders of domestic female pigs (˜25 kg) that have undergone complete cystectomy (at the trigon region) under aseptic conditions throughout the process. The specimens were excised in an operating room under aseptic conditions. Each cyst was washed with saline and placed in a sterile cup (filled with saline) on ice till delivery to the lab. The samples were removed from the cups inside a biosafety cabinet with ventilation turned on after thorough disinfection of each cup with 70% alcohol. Each bladder was cut in half (dome to trigon direction) and washed several times (at least 5) for a few minutes (1-3) in saline to remove as much blood as possible. Each part was then placed on a clean culture dish and the mucosa was microsurgically separated from the remaining tissue using forceps and microscissors or scalpel. Each half was then stretched on soft plastic molds (3D printed molds, homemade), immobilized with insect pins (urothelial side facing up), and placed inside a new culture dish (10 cm diameter). The dish was immediately filled with 20 ml of 1.5-4 mg/ml Dispase II solution in HBSS without magnesium and calcium and incubated at 4° C. for 8-16 hours. After the incubation, the Dispase II solution was discarded and incubated with 20 ml of Trypsin-EDTA solution for 30 min at 37° C. (cell culture incubator). Then, the tissue was unpinned and placed in a new culture dish with the mucosal side facing the lid. The dish was filled with 15 ml of PBS solution supplemented with 2-5% v/v FBS. The urothelial cells were scraped off as cell sheets with a scalpel blade and the remaining tissue was discarded. The cell sheets were then dissociated in single cells mechanically using a 10 ml pipette (several up-down with the pipette tip facing firmly on the culture dish while pushing downwards the solution). Dissociation progress was evaluated by optical means (microscope). The cells were then passed through a 40 μm cell strainer and collected in a 50 ml tube. The tube was filled up to 45 ml with additional PBS-FBS and centrifuged at 1500 rpm for 5 min. The pellet was washed once more with another 45 ml of PBS-FBS and resuspended at the desired volume of KSFM medium. For direct cryopreservation of the cells about 4-8×10⁶ cells per cryovial were frozen (marked as passage 0) in appropriate cryopreservative media (50% KSFM, 40% FBS, and 10% DMSO), while for expansion of the isolated cells, 3-6×10⁶ cells were seeded in 10 cm culture dish in KSFM (marked as passage 0) with the addition of antibiotic mixture (Gentamicin, Amphotericin B and Ciprofloxacin). Human pUCs may be isolated with the same protocol but with shorted Dispase II digestion (1-3 hours depending on the sample size).

Primary stromal cells are isolated as tissue explants. Small fragments of the whitish tissue between the urothelium and the red muscular bladder wall are microsurgically isolated after the separation of the bladder mucosa from the incised bladder tissue and placed in a culture dish aseptically. The pieces are chopped down to miniature pieces (less than 1 mm) and placed densely on the bottom of a culture dish (˜100 pieces per 10 cm in diameter dish). The miniature pieces were allowed to dry and adhere on the plate for ˜30 min in the biosafety cabinet prior to adding 10 ml of complete DMEM medium with antibiotics (Gentamicin, Amphotericin B, and Ciprofloxacin). The medium was changed every 2-3 days until explant outgrowth reached 70-80% confluence. Then the cells were either cryopreserved or passaged for expansion.

### Example 2. In Vitro Expansion

Cryopreserved or freshly isolated porcine pUCs are cultured in KSFM medium. One cryovial or 4-6×10⁶ freshly isolated cells were seeded in a 10 cm culture dish in 10 ml of KSFM with the addition of an antibiotic mixture (Gentamicin, Amphotericin B, and Ciprofloxacin). For the first two passages, the medium was supplemented with 2-5% FBS. The cells were passaged at 70-80% confluence after trypsinization. Growth medium was replaced every other day. The optimal split ratio for porcine pUCs is 1:4. The cells appear with epithelial morphology and form small colonies (6-10 cells in size) the day after plating. These colonies expand, reach, and combine neighboring ones as they grow. The cells progressively lose their initial growth rate (reaching confluence at ˜2 days in culture) and sporadic senescent cells become more frequent after 4-5 passages. The cells at passage 2 express keratin 5 and keratin 14. Cells of passage 1 or 2 were utilized for experiments. For ex vivo differentiation and urothelial stratification, pUCs were exposed to increased concentrations of calcium by supplementing the growth medium with 2-6 M of CaCl₂.

An alternative way of expanding and differentiating the pUCs is to encapsulate them on 3D extracellular matrices like Matrigel and BME (basement membrane extract) in order to form organoids. Such organoids can be printed instead of pUCs in this in vivo bioprinting application.

As used herein, an “organoid” is a three-dimensional miniaturized version of an organ that can be produced in vitro from one or more stem cells that organize into larger cell organizations and which mimic some properties of a particular human organ. Organoids may be used for screening new drug candidates as the receptors on the cells of organoids often behave more like the receptors on the cells of the corresponding human organs than the receptors on the cells of animal models. Human pUCs are expanded in the same manner as porcine ones but without any FBS in their media.

### Example 3. Cystoplasty Surgical Procedure (Bladder)

Domestic female pigs (˜25 kg) are used. The animals were allowed a week for acclimatization with free access to food and water. The day before surgery they were fasted, allowing only water consumption. Prior to surgery, the animals were pre-anesthetized via an intramuscular (IM.) injection of ketamine (10 mg/kg body weight, Ketamidor, Richter pharma AG, Austria) and midazolam 0.5 mg/kg body weight; Dormicum; Roche, Athens, Greece). An intravenous catheter (BD Venflon 20G, 10×32 mm) was placed and secured at the auricular vein for parenteral administration of propofol and antibiotics. Suppression for the intubation procedure was achieved by bolus administration of propofol 1% at 1-1.4 mg/kg. Each intubation was carried out using a conventional laryngoscope with a metal bent blade via mouth-tracheal access using 6.0-6.5 mm cuffed tracheal tubes. For analgesia Meloxicam (Melovem 5 mg/ml) was administered IM at a dose of 0.4 mg/kg and antibiotic cephalosporin 25 mg/kg (zinacef 750 mg) was administered intravenously. Pigs were put on mechanical ventilation throughout the surgical procedure and anesthesia was maintained with inhalant isoflurane at a concentration range of 2.5-3.5% and an oxygen flow rate at 1.5 lit/min. Vital signs (heart rate and SPO2) were monitored throughout the procedure. Next, they were prepared for surgery with leg strapping on the surgical table, disinfection of the abdomen, and fitting of appropriately sized surgical screens.

The process starts with an abdominal incision (diathermy) of about 11 cm at the midline on the lower abdomen. 40 cm away from the ileocecal valve, a 30 cm intestinal part was isolated with its mesentery intact. This part was sealed in warm wet gauze (prewarmed saline at 37 degrees Celsius) until the intestine was anastomosed (end-to-end) with 3.0 PDS absorbable stitches. Then the 30 cm incised intestine was cut longitudinally at the antimesenteric side with scissors with the surgeon holding one side and the assistant the other, at 5 cm steps. After washing the exposed luminal part with warm saline and gauges, the surgeon separates the mucosal part from the remaining tissue (mainly muscularis externae and part of the lamina propria). Initially, the muscular part is held with a large Debakiy forceps and the mucosal is separated with a smaller one. Once the first 2-3 cm of the intestinal mucosa is separated the muscular tissue can be held with hands as the manual mechanical denudation proceeds until complete separation throughout the intestinal segment is achieved. The graft is now ready for in vivo bioprinting, which includes the steps of denuding (e.g., via chemical or laser 402 means) intestinal tissue and bioprinting onto the resultant denuded intestinal tissue via LIFT, as shown in FIG. 8).

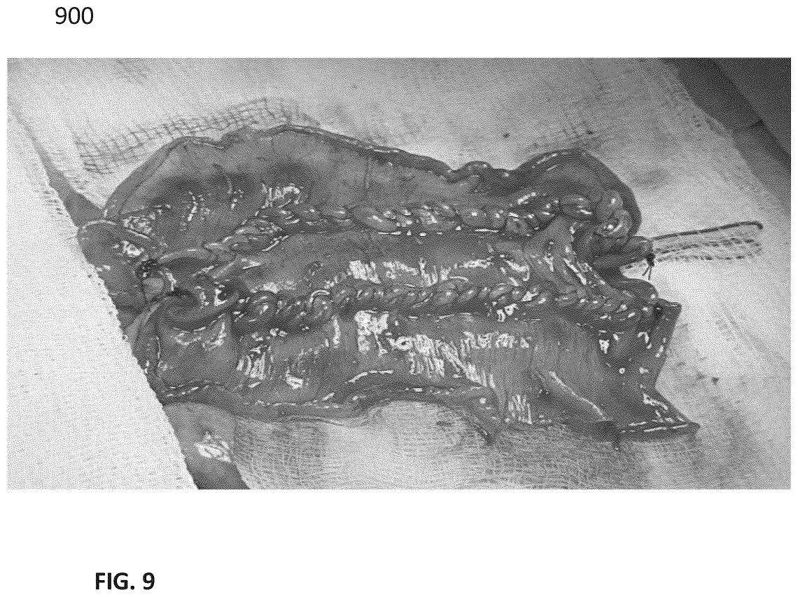

After bioprinting (e.g., step 106), the segment is placed in an “S” shape and the opposing ends are sutured with 3.0 PDS absorbable stitches, as shown in the sutured tissue 900 in FIG. 9. The resultant graft (˜8×10 cm) is then stretched with the aid of six musket forceps (three on each side). At this stage, either an appropriately sized GAG layer is prepared (photopolymerized in a mold) and sutured on the graft (on top of printed pUCs or is also LIFT printed on top of pUCs and photopolymerized in situ. The graft is now ready to replace the bladder.

Prior to grafting, the ureters are identified and excised 1 cm away from the bladder. catheters are inserted (one on each ureter) and the ureters are sutured parallel on the graft (˜1.5 cm away from the short edge of the graft and at ˜4 cm distance between them). The native bladder is excised just below the ureters. The short side of the graft with the nearby anastomosed ureters is then placed in parallel to the remaining bladder base and it is sutured with 4.0 PDS absorbable stitches starting from one corner of the graft all around till meeting the first suture. Then, the catheters are inserted into the urethra in the direction of urine flow till exiting the body. A 3-way Foley Dufur tip catheter (18Ch) is inserted with the aid of a lubricant. Next, the suturing continues to the long sides of the graft and concludes with the other small side on top. Through the catheter, 10-30 ml of warm saline is slowly injected into the neobladder to assess possible leaking points. If such points are identified, they are sealed with additional stitches. The saline is removed, and the Foley balloon is filled slowly with 15-25 ml of warm saline to preserve the neobladder's spherical shape and to avoid symphysis (opposing ends of the neobladder to adhere). The surgery resumes with washing the internal organs and suturing the abdomen (layer by layer).

Of note, the pUC bioprinting (e.g., step 106) can be also performed after reshaping (e.g., shaping) the denuded intestine into “S”. It is also possible to print pUCs onto photopolymerized GAG of an appropriate size and sutured on the “S” shaped denuded intestine just prior to grafting to the excised bladder. Any of the abovementioned bioprinting setups can be performed with pUC-derived organoids instead of single cells (pUCs). A similar procedure can be followed in humans with appropriate adjustments (e.g. pre-/post-operation care i.e. restrictions/medications etc, anesthesia, catheter/suture sizes, etc).

### Example 4. Cystoplasty Surgical Procedure (Urethroplasty) and Urethral Repair

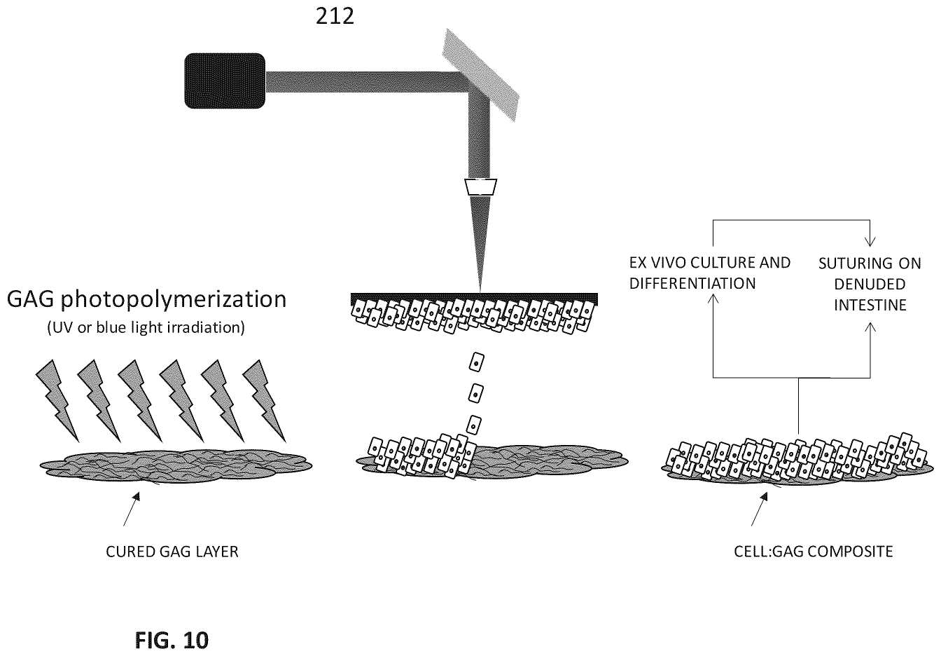

The typical urethroplasty procedure for large stricture (sized >2 cm) can be modified in the part that no autologous tissue (oral mucosa, scrotal graft, or penile island flap) will be grafted but in their position pUCs will be printed on the site of the excised urethra. Alternatively, a GAG scaffold previously prepared or molded around a catheter of appropriate size (the use of which is mandatory in all urethroplasties) can be generated and autologous pUCs will be printed on it just prior to grafting (FIG. 10). In either way, a new urethra will emerge from the expansion and differentiation of printed pUCs.

### Example 5. Laser Printing of UCs In-Vivo on the Denuded Intestine

The laser beam (e.g., the LIFT beam 504) exits the laser unit (e.g., the laser 502), and is guided through an optical path, a configuration of optics and lenses, before being focused at the interface between the donor plate and the material to be transferred (FIG. 10). In our experiments, the printing speed that was used is 1 m/s. The camera of the imaging system 730 is coupled with a magnifying system, resulting in a total optical magnification of 5×.

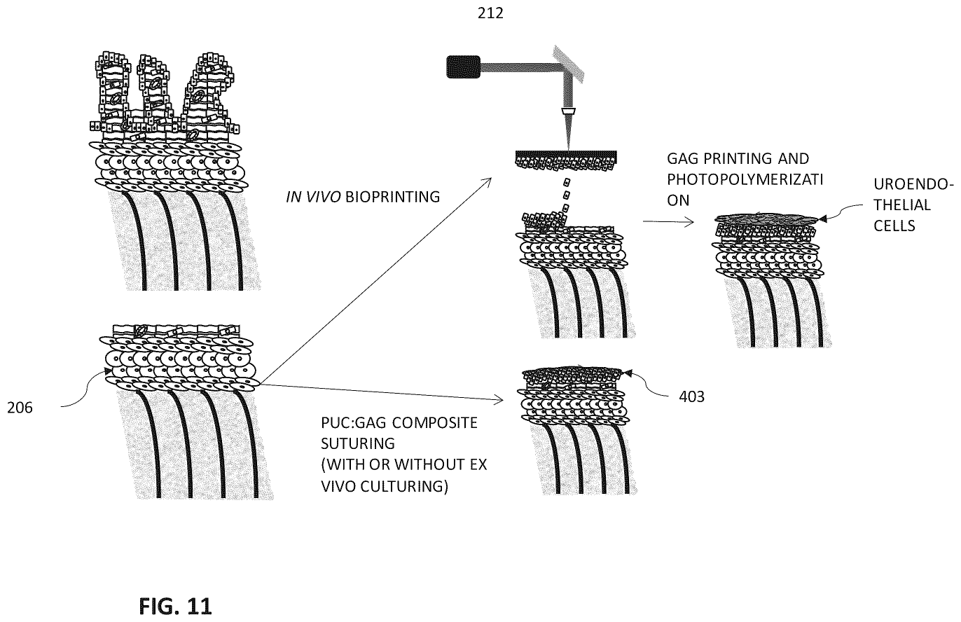

Printing of urothelial cells onto the denuded intestinal tissue was performed as detailed herein. The preparation of the denuded intestinal tissue, is described in Example 3, where a small section of the intestinal tissue 2-3 cm long was processed for the in vivo LIFT printing of UCs. All LIFT experiments are performed at laser fluences between 50 and 500 mJ/cm², while the energy of the projected laser beam is controlled via an attenuator plate. Referring to FIG. 8 (e.g., the in vivo bioprinting principle), LIFT printing of the cell suspension (50-150×10³ pUCs dissolved in growth medium) is performed at 1 KHz repetition rate. The gap between the donor substrate and the denuded intestinal tissue can be adjusted and is set to a value in the range of 100 to 10000 μm, while the resulting printed droplet diameter is in the range of 30 to 300 μm. The resulting droplet size of the transferred cell suspension can be adapted by tuning the LIFT printing processing conditions, such as the laser pulse energy (calculated by dividing the average power with the laser repetition rate), the distance between donor substrate and the receiver substrate, the focused laser spot size, as well as the thickness of the cell suspension layer. Referring to FIG. 11 (e.g., illustrating neobladder formation through intestine de-epithelialization and in vivo printing), about 200M cells were printed in about 2-10 minutes on the denuded intestinal tissue.

This method may also be used to LIFT print soluble GAG on top of pUCs after reforming the printed denuded intestine into an “S” shaped graft as described in Example 3. The laser unit will be selected, depending on the curation conditions for the GAG layer preparation as described in Example 6 and shown in FIG. 11.

### Example 6. Printing of UCs on GAG (Ex-Vivo) and Transplantation on the Denuded Intestine

The glucosaminoglycan (GAG) layer on the luminal side of the umbrella cells aids the waterproof blood urine barrier and consists mainly of hyaluronic acid (HA) and chondroitin sulfate (CS). Both of them are commercially available and can be easily modified to become methacrylated (doi.org/10.1016/j.biomaterials.2020.120602) or purchased as methacrylated (MA). MA-HS, MA-CS, or mixtures of them in 1:4 up to 4:1 ratios can be utilized to form an acellular matrix that can be photopolymerized with UV or green light, depending on the photoinitiator utilized (e.g., Irgacure 2959 or Lithium phenyl-2,4,6-trimethylbenzoylphosphinate-LAP), for few seconds up to several minutes depending on the desired thickness and stiffness of the end product. This GAG-mimicking matrix can be utilized in several ways. One is to prepare a GAG layer of the size of the graft (in a mold) and after photopolymerization to suture it on top of the printed pUCs on the denuded intestine to serve as a luminal protective waterproof layer for pUCs (FIG. 11). Another is to prepare a similar sized GAG layer and after curation to print there the pUCs (instead of denuded intestine) and suture this composite onto “S” shaped denuded intestine (FIG. 10 and FIG. 11). A GAG layer can also be prepared in a culture dish and used as a scaffold that pUCs can be printed onto and cultured there until differentiate into a stratified urothelium. Then, this composite can be sutured on the denuded intestinal segment prior to grafting onto the bladder as an alternative method to the classic augmentation or complete enterocystoplasty (FIG. 10 and FIG. 11). Assessment of bioprinting can be assessed post-printing via immunofluorescence. These alternative approaches can be utilized in both pigs and humans.

In a similar manner, tubular cellularized GAG scaffolds can be grafted in urethroplasty procedures replacing typical transplants like oral mucosa or genital tissue. In this case, a thin (up to 2 mm) GAG scaffold can be generated, in situ (around a catheter) or in the lab (homemade mold). This tubular scaffold will be then cellularized (autologous pUCs) through LIFT bioprinting on its outer surface. The cellularized GAG scaffold may be generated and used in situ (as part of our new urethroplasty approach) or cultured in vitro prior to grafting.

The present invention is further described in the following numbered paragraphs:

1. A method for in-vivo cell replacement including:
extracting donor cells from a donor tissue of a patient;
generating donor material including the donor cells;
removing one or more cell layers from a receiver tissue of the patient to expose a target layer of the receiver tissue while maintaining operation of a vasculature of the receiver tissue; and
printing the donor material onto the target layer of the receiver tissue.

2. The method of paragraph 1, wherein generating the donor material includes: expanding the donor cells to create additional donor cells.

3. The method of paragraph 1, wherein removing the one or more cell layers from the receiver tissue of the patient to expose the target layer of the receiver tissue without disrupting the vasculature of the receiver tissue includes:
removing the one or more cell layers using at least one of laser ablation, mechanical separation, chemical separation, or enzymatic separation.

4. The method of paragraph 1, wherein printing the donor material includes: printing the donor material using laser induced forward transfer (LIFT).

5. The method of paragraph 1, wherein printing the donor material includes: printing the donor material using at least one of an extrusion or an inkjet process.

6. The method of paragraph 1, further including:
verifying that the donor material is free of cancer cells.

7. The method of paragraph 1, further including:
grafting the receiver tissue to an additional tissue of the patient while maintaining the operation of a vasculature of the receiver tissue.

8. The method of paragraph 7, further including:
shaping the receiver tissue to a desired shape using sutures prior to grafting the receiver tissue to the additional tissue.

9. The method of paragraph 7, further including:
shaping the receiver tissue to a desired shape using sutures prior to printing the donor material.

10. The method of paragraph 1, wherein the donor material includes at least one of urothelial cells, stromal cells, or epithelial cells.

11. The method of paragraph 1, further including:
placing additional material on the target layer of the receiver tissue prior to printing the donor material onto the target layer of the receiver tissue, wherein printing the donor material onto the target layer of the receiver tissue includes:
printing the donor material onto the additional material.

12. The method of paragraph 11, wherein the additional material includes:
at least one of platelet-rich plasma (PRP) or polymerized acellular glucosaminoglycan (GAG).

13. The method of paragraph 1, wherein the donor material includes urothelial cells, wherein the receiver tissue includes intestinal tissue, wherein removing the one or more cell layers from the receiver tissue of the patient to expose the target layer of the receiver tissue without disrupting the vasculature of the receiver tissue includes removing epithelial cells from the intestinal tissue, wherein the method further includes grafting the intestinal tissue to a bladder of the patient.

14. The method of paragraph 1, wherein the donor material includes urothelial cells, wherein the receiver tissue includes a urethra, wherein removing the one or more cell layers from the receiver tissue of the patient to expose the target layer of the receiver tissue without disrupting the vasculature of the receiver tissue includes removing one or more damaged portions of the urethra.

15. The method of paragraph 1, wherein the donor material includes esophageal keratinocytes, wherein the receiver tissue includes intestinal tissue, wherein removing the one or more cell layers from the receiver tissue of the patient to expose the target layer of the receiver tissue without disrupting the vasculature of the receiver tissue includes removing epithelial cells from the intestinal tissue, wherein the method further includes grafting the intestinal tissue to an esophagus of the patient.

16. The method of paragraph 13, wherein an additional tissue of a patient includes: a urethra of the patient.

17. The method of paragraph 1, further including:
applying at least one of polymerized acellular glucosaminoglycan (GAG) or platelet-rich plasma (PRP) to the receiver tissue after printing the donor material.

18. The method of paragraph 1, wherein the patient is a non-human animal.

19. A method including:
extracting donor cells from a donor tissue of a patient;
generating donor material including the donor cells;
removing one or more cell layers from a receiver tissue of the patient to expose a target layer of the receiver tissue while maintaining operation of a vasculature of the receiver tissue;
printing the donor material including urothelial cells onto an intermediate layer; and
placing the intermediate layer with the donor material onto the target layer of the receiver tissue in the patient.

20. The method of paragraph 19, wherein the intermediate layer includes:
at least one of polymerized acellular glucosaminoglycan (GAG) or platelet-rich plasma (PRP).

21. The method of paragraph 19, wherein generating the donor material includes: expanding the donor cells to create additional donor cells.

22. The method of paragraph 19, wherein removing the one or more cell layers from the receiver tissue of the patient to expose the target layer of the receiver tissue without disrupting the vasculature of the receiver tissue includes:
removing the one or more cell layers using at least one of laser ablation, mechanical separation, chemical separation, or enzymatic separation.

23. The method of paragraph 19, wherein printing the donor material includes: printing the donor material using laser induced forward transfer (LIFT).

24. The method of paragraph 19, wherein printing the donor material includes: printing the donor material using at least one of an extrusion or an inkjet process.

25. The method of paragraph 19, wherein including:
verifying that the donor material is free of cancer cells.

26. The method of paragraph 19, wherein including:
grafting the receiver tissue to an additional tissue of the patient while maintaining the operation of the vasculature of the receiver tissue.

27. The method of paragraph 26, further including:
shaping the receiver tissue to a desired shape using sutures prior to grafting the receiver tissue to the additional tissue.

28. The method of paragraph 26, further including:
shaping the receiver tissue to a desired shape using sutures prior to printing the donor material.

29. The method of paragraph 19, wherein the donor material includes at least one of urothelial cells, stromal cells, or epithelial cells.

30. The method of paragraph 19, further including:
placing additional material on the target layer of the receiver tissue prior to printing the donor material onto the target layer of the receiver tissue, wherein printing the donor material onto the target layer of the receiver tissue includes:
printing the donor material onto the additional material.

31. The method of paragraph 30, wherein the additional material includes:
at least one of platelet-rich plasma (PRP) or polymerized acellular glucosaminoglycan (GAG).

32. The method of paragraph 19, wherein the donor material includes the urothelial cells, wherein the receiver tissue includes intestinal tissue, wherein removing the one or more cell layers from the receiver tissue of the patient to expose the target layer of the receiver tissue without disrupting the vasculature of the receiver tissue includes removing epithelial cells from the intestinal tissue, wherein the method further includes grafting the intestinal tissue to a bladder of the patient.

33. The method of paragraph 19, wherein the donor material includes the urothelial cells, wherein the receiver tissue includes a urethra, wherein removing the one or more cell layers from the receiver tissue of the patient to expose the target layer of the receiver tissue without disrupting the vasculature of the receiver tissue includes removing one or more damaged portions of the urethra.

34. The method of paragraph 19, wherein the donor material includes esophageal keratinocytes, wherein the receiver tissue includes intestinal tissue, wherein removing the one or more cell layers from the receiver tissue of the patient to expose the target layer of the receiver tissue without disrupting the vasculature of the receiver tissue includes removing epithelial cells from the intestinal tissue, wherein the method further includes grafting the intestinal tissue to an esophagus of the patient.

35. The method of paragraph 32, wherein an additional tissue of the patient includes: a urethra of the patient.

36. The method of paragraph 19, further including:
applying at least one of polymerized acellular glucosaminoglycan (GAG) or platelet-rich plasma (PRP) to the receiver tissue after printing the donor material.

37. The method of paragraph 19, wherein the patient is a non-human animal.

38. An augmented bladder or orthotopic neobladder including:
a first portion of a bladder of a patient; and
a graft formed from intestinal tissue of the patient with undisturbed vasculature, wherein the intestinal tissue includes a layer of donor material including urothelial cells from the patient printed by steps of:
extracting donor cells from a donor tissue of a patient;
generating the donor material including the donor cells;
removing one or more cell layers from the intestinal tissue of the patient to expose a target layer of the intestinal tissue while maintaining operation of a vasculature of the intestinal tissue; and
printing the donor material onto the target layer of the intestinal tissue.

39. The augmented bladder or orthotopic neobladder of paragraph 38, further including: placing additional material on the target layer of the intestinal tissue prior to printing the donor material onto the target layer of the intestinal tissue, wherein printing the donor material onto the target layer of the intestinal tissue includes:
printing the donor material onto the additional material.

40. The augmented bladder of paragraph 39, wherein the additional material includes: at least one of platelet-rich plasma (PRP) or polymerized acellular glucosaminoglycan (GAG).

41. The augmented bladder of paragraph 38, wherein removing the one or more cell layers from a receiver tissue of the patient to expose the target layer of the receiver tissue without disrupting vasculature of the receiver tissue includes:
removing the one or more cell layers using at least one of laser ablation, mechanical separation, chemical separation, or enzymatic separation.

42. The augmented bladder of paragraph 38, wherein printing the donor material includes: printing the donor material using laser induced forward transfer (LIFT).

43. The augmented bladder of paragraph 38, wherein printing the donor material includes: printing the donor material using at least one of an extrusion or an inkjet process.

44. The augmented bladder of paragraph 38, wherein the patient is a non-human animal.

45. A tissue position assembly including:
a rail formed from a sterilizable material, wherein the rail is configured to connect to a replaceable receiver assembly, the replaceable receiver assembly further configured to secure and position a tissue of a patient while maintaining operation of vasculature of the tissue, wherein the rail is further configured to connect to a replaceable donor assembly, the replaceable donor assembly configured to secure and position a donor plate with donor material on a surface facing the replaceable receiver assembly; and
a housing connected to the rail, wherein the housing is configured to secure one or more lenses configured to focus laser light from a laser source onto the donor plate to transfer a portion of the donor material to receiver material.

46. The tissue position assembly of paragraph 45, wherein the rail is configured to connect to the replaceable donor assembly via a carriage, wherein the carriage is positionable at one or more locations along the rail to provide one or more corresponding separation distances between the donor plate connected to a replaceable donor assembly and the tissue secured to the replaceable receiver assembly.

47. The tissue position assembly of paragraph 46, further including:
a detector configured to determine a thickness of the tissue secured to the replaceable receiver assembly in a direction of the donor plate.

48. The tissue position assembly of paragraph 47, wherein a separation distance between the donor plate and the tissue secured to the replaceable receiver assembly is adjusted based on the thickness of the tissue.

49. The tissue position assembly of paragraph 47, wherein the detector includes: an imaging system providing a fixed working distance and secured on a translation stage, wherein the detector determines the thickness of the tissue based on images generated at different a position of the imaging system on the translation stage providing an in-focus image of the tissue.

50. The tissue position assembly of paragraph 46, wherein the carriage is formed from the sterilizable material.

51. The tissue position assembly of paragraph 46, wherein the carriage is disposable.

52. The tissue position assembly of paragraph 46, wherein the housing is sealed, wherein the housing includes one or more windows transparent to the laser light for coupling the laser light at least one of into or out of the housing.

53. The tissue position assembly of paragraph 45, wherein the rail is formed from a metal.

54. The tissue position assembly of paragraph 53, wherein the metal includes: at least one of aluminum or stainless steel.

55. The tissue position assembly of paragraph 45, wherein at least one of the replaceable receiver assembly or the replaceable donor assembly is formed from a biocompatible material.

56. The tissue position assembly of paragraph 46, wherein biocompatible material includes:
at least one of polyurethane, polycarbonate, polyethylene, or polypropylene.

57. A composition for use in a treatment of bladder damage or bladder dysmorphism including:
donor material containing cells suitable for transfer from a donor substrate to receiver material via a bioprinter;
media suitable for transfer of the donor material from the donor substrate to the receiver material; and
the donor substrate loaded with the donor material and the media.

58. The composition of paragraph 57, the donor substrate including:
a glass substrate layer; and
a metallic film layer.

59. The composition of paragraph 57, wherein the donor material includes primary cells.

60. The composition of paragraph 59, wherein the cells include induced pluripotent cells (IPS).

61. The composition of paragraph 59, wherein the cells include differentiated cells derived from iPS cells.

62. The composition of paragraph 59, wherein the cells include urothelial cells.

63. The composition of paragraph 57, wherein the donor material further includes polymerized acellular glucosaminoglycan (GAG).

64. The composition of paragraph 57, wherein the media includes KSFM medium.

65. The composition of paragraph 57, wherein the donor substrate includes organoids.

66. The composition of paragraph 65, wherein the organoids include primary urothelial cells.

67. A system for supplying donor material to a bioprinter including:
a donor frame;
an automated stage configured to move the donor frame along an X-axis and a Y-axis;
a dispenser subsystem including a pump and configured to deliver transport donor material from a reservoir to a material feeding head;
one or more controllers communicatively coupled to the automated stage and the dispenser subsystem, the one or more controllers including one or more processors configured to execute a set of program instructions stored in a memory, the set of program instructions configured to cause the one or more processors to:
operate the pump, wherein operating the pump transports donor material from the reservoir through the material feeding head; and
operate the automated stage to move the donor frame in coordination with an operation of the pump, wherein the donor material is transported to a designated area via a movement of the donor frame and the operation of the pump.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected” or “coupled” to each other to achieve the desired functionality, and any two components capable of being so associated can also be viewed as being “couplable” to each other to achieve the desired functionality. Specific examples of couplable include but are not limited to physically interactable and/or physically interacting components and/or wirelessly interactable and/or wirelessly interacting components and/or logically interactable and/or logically interacting components.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A method for in-vivo cell replacement comprising:
extracting donor cells from a donor tissue of a patient;
generating donor material including the donor cells;
removing one or more cell layers from a receiver tissue of the patient to expose a target layer of the receiver tissue while maintaining operation of a vasculature of the receiver tissue; and
printing the donor material onto the target layer of the receiver tissue.

2. The method of claim 1, wherein generating the donor material comprises:
expanding the donor cells to create additional donor cells.

3. The method of claim 1, wherein removing the one or more cell layers from the receiver tissue of the patient to expose the target layer of the receiver tissue without disrupting the vasculature of the receiver tissue comprises:
removing the one or more cell layers using at least one of laser ablation, mechanical separation, chemical separation, or enzymatic separation.

4. The method of claim 1, wherein printing the donor material comprises:
printing the donor material using laser induced forward transfer (LIFT).

5. The method of claim 1, wherein printing the donor material comprises:
printing the donor material using at least one of an extrusion or an inkjet process.

6. The method of claim 1, further comprising:
grafting the receiver tissue to an additional tissue of the patient while maintaining the operation of a vasculature of the receiver tissue.

7. The method of claim 1, wherein the donor material includes at least one of urothelial cells, stromal cells, or epithelial cells.

8. The method of claim 1, wherein the donor material comprises urothelial cells, wherein the receiver tissue comprises intestinal tissue, wherein removing the one or more cell layers from the receiver tissue of the patient to expose the target layer of the receiver tissue without disrupting the vasculature of the receiver tissue comprises removing epithelial cells from the intestinal tissue, wherein the method further comprises grafting the intestinal tissue to a bladder of the patient.

9. The method of claim 1, wherein the donor material comprises urothelial cells, wherein the receiver tissue comprises a urethra, wherein removing the one or more cell layers from the receiver tissue of the patient to expose the target layer of the receiver tissue without disrupting the vasculature of the receiver tissue comprises removing one or more damaged portions of the urethra.

10. The method of claim 8, wherein an additional tissue of a patient comprises:
a urethra of the patient.

11. The method of claim 1, further comprising:
applying at least one of polymerized acellular glucosaminoglycan (GAG) or platelet-rich plasma (PRP) to the receiver tissue after printing the donor material.

12. The method of claim 1, wherein the patient is a non-human animal.

13. An augmented bladder comprising:
a first portion of a bladder of a patient; and
a graft formed from intestinal tissue of the patient with undisturbed vasculature, wherein the intestinal tissue includes a layer of donor material including urothelial cells from the patient printed by steps of:
extracting donor cells from a donor tissue of a patient;
generating the donor material including the donor cells;
removing one or more cell layers from the intestinal tissue of the patient to expose a target layer of the intestinal tissue while maintaining operation of a vasculature of the intestinal tissue; and
printing the donor material onto the target layer of the intestinal tissue.

14. The augmented bladder of claim 13, further comprising:
placing additional material on the target layer of the intestinal tissue prior to printing the donor material onto the target layer of the intestinal tissue, wherein printing the donor material onto the target layer of the intestinal tissue comprises:
printing the donor material onto the additional material.

15. The augmented bladder of claim 14, wherein the additional material comprises:
at least one of platelet-rich plasma (PRP) or polymerized acellular glucosaminoglycan (GAG).

16. The augmented bladder of claim 13, wherein removing the one or more cell layers from a receiver tissue of the patient to expose the target layer of the receiver tissue without disrupting vasculature of the receiver tissue comprises:
removing the one or more cell layers using at least one of laser ablation, mechanical separation, chemical separation, or enzymatic separation.

17. The augmented bladder of claim 13, wherein printing the donor material comprises:
printing the donor material using laser induced forward transfer (LIFT).

18. The augmented bladder of claim 13, wherein printing the donor material comprises:
printing the donor material using at least one of an extrusion or an inkjet process.

19. The augmented bladder of claim 13, wherein the patient is a non-human animal.

20. A tissue position assembly comprising:
a rail formed from a sterilizable material, wherein the rail is configured to connect to a replaceable receiver assembly, the replaceable receiver assembly further configured to secure and position a tissue of a patient while maintaining operation of vasculature of the tissue, wherein the rail is further configured to connect to a replaceable donor assembly, the replaceable donor assembly configured to secure and position a donor plate with donor material on a surface facing the replaceable receiver assembly; and
a housing connected to the rail, wherein the housing is configured to secure one or more lenses configured to focus laser light from a laser source onto the donor plate to transfer a portion of the donor material to receiver material.

21. The tissue position assembly of claim 20, wherein the rail is configured to connect to the replaceable donor assembly via a carriage, wherein the carriage is positionable at one or more locations along the rail to provide one or more corresponding separation distances between the donor plate connected to a replaceable donor assembly and the tissue secured to the replaceable receiver assembly.

22. The tissue position assembly of claim 21, further comprising:
a detector configured to determine a thickness of the tissue secured to the replaceable receiver assembly in a direction of the donor plate.

23. The tissue position assembly of claim 22, wherein a separation distance between the donor plate and the tissue secured to the replaceable receiver assembly is adjusted based on the thickness of the tissue.

24. The tissue position assembly of claim 22, wherein the detector comprises:
an imaging system providing a fixed working distance and secured on a translation stage, wherein the detector determines the thickness of the tissue based on images generated at different a position of the imaging system on the translation stage providing an in-focus image of the tissue.

25. The tissue position assembly of claim 21, wherein the carriage is formed from the sterilizable material.

26. The tissue position assembly of claim 21, wherein the carriage is disposable.

27. The tissue position assembly of claim 21, wherein the housing is sealed, wherein the housing includes one or more windows transparent to the laser light for coupling the laser light at least one of into or out of the housing.

28. The tissue position assembly of claim 20, wherein the rail is formed from a metal.

29. The tissue position assembly of claim 28, wherein the metal comprises:
at least one of aluminum or stainless steel.

30. The tissue position assembly of claim 20, wherein at least one of the replaceable receiver assembly or the replaceable donor assembly is formed from a biocompatible material.

31. The tissue position assembly of claim 21, wherein biocompatible material comprises:
at least one of polyurethane, polycarbonate, polyethylene, or polypropylene.

32. A composition for use in a treatment of bladder damage or bladder dysmorphism comprising:
donor material containing cells suitable for transfer from a donor substrate to receiver material via a bioprinter;
media suitable for transfer of the donor material from the donor substrate to the receiver material; and
the donor substrate loaded with the donor material and the media.

33. The composition of claim 32, the donor substrate comprising:
a glass substrate layer; and
a metallic film layer.

34. The composition of claim 32, wherein the donor material comprises primary cells.

35. The composition of claim 34, wherein the cells comprise induced pluripotent cells (iPS).

36. The composition of claim 34, wherein the cells comprise differentiated cells derived from iPS cells.

37. The composition of claim 34, wherein the cells comprise urothelial cells.

38. The composition of claim 32, wherein the donor material further comprises polymerized acellular glucosaminoglycan (GAG).

39. The composition of claim 32, wherein the media comprises KSFM medium.

40. The composition of claim 32, wherein the donor substrate comprises organoids.

41. The composition of claim 40, wherein the organoids comprise primary urothelial cells.

42. A system for supplying donor material to a bioprinter comprising:
a donor frame;
an automated stage configured to move the donor frame along an X-axis and a Y-axis;
a dispenser subsystem comprising a pump and configured to deliver transport donor material from a reservoir to a material feeding head;
one or more controllers communicatively coupled to the automated stage and the dispenser subsystem, the one or more controllers including one or more processors configured to execute a set of program instructions stored in a memory, the set of program instructions configured to cause the one or more processors to:
operate the pump, wherein operating the pump transports donor material from the reservoir through the material feeding head; and
operate the automated stage to move the donor frame in coordination with an operation of the pump, wherein the donor material is transported to a designated area via a movement of the donor frame and the operation of the pump.
